(19) United States
(12) Patent Application Publication
(10) Pub. No.: US 2021/0259560 A1
(43) Pub. Date: Aug. 26, 2021

(54) METHODS AND SYSTEMS FOR DETERMINING A PHYSIOLOGICAL OR BIOLOGICAL STATE OR CONDITION OF A SUBJECT

(71) Applicant: Eko Devices, Inc., Oakland, CA (US)

(72) Inventors: Subramaniam Venkatraman, Oakland, CA (US); John Maidens, Oakland, CA (US); Avi Shapiro, Oakland, CA (US); John Prince, Oakland, CA (US); Steve L. Pham, Oakland, CA (US); Connor Landgraf, Oakland, CA (US)

(21) Appl. No.: 17/187,344

(22) Filed: Feb. 26, 2021

Related U.S. Application Data
(60) Provisional application No. 62/982,000, filed on Feb. 26, 2020.

(51) Int. Cl. A61B 5/024; A61B 5/00; A61B 5/02; A61B 5/318; A61B 5/28

(52) U.S. Cl. CPC A61B 5/024; A61B 5/0006; A61B 5/02028; A61B 5/318; A61B 2562/0219; A61B 5/7264; A61B 5/0022; A61B 5/6898; A61B 5/28

(57) ABSTRACT

The present disclosure provides methods, devices, and systems for determining a state or condition of a subject. A method for determining a state or condition of a heart of a subject may include using a monitoring device comprising a plurality of sensors comprising an electrocardiogram (ECG) sensor, an audio sensor, and other sensors to measure data including ECG data and audio data from an organ of the subject and transmitting the data wirelessly to a computing device. A trained algorithm may be used to process the data, such as the ECG data, the audio data, and other data to determine the state or condition of the organ of the subject. More specifically, the trained algorithm can be customized for a specific indication or condition. An output indicative of the state or condition of the heart of the subject may be provided on the computing device or monitoring device.

### CROSS-REFERENCE TO RELATED APPLICATIONS

The present application claims priority to U.S. Provisional Application No. 62/982,000 entitled “METHODS AND SYSTEMS FOR DETERMINING A PHYSIOLOGICAL OR BIOLOGICAL STATE OR CONDITION OF A SUBJECT”, and filed on Feb. 26, 2021. The entire contents of the above-listed application are hereby incorporated by reference for all purposes.

### FIELD

The present description relates generally to methods and systems for a digital health monitoring device.

### BACKGROUND/SUMMARY

As healthcare costs continue to escalate, solutions to reduce the cost and increase the efficacy of diagnostic efforts may become increasingly desired. In other situations, increasing access to medical diagnostic and monitoring capabilities may be desirable. These objectives may be particularly valuable for cardiac care, since cardiac function is central to human health and well-being, and cardiovascular diseases (CVDs) continue to be the most common cause of death. Such cardiovascular diseases may include coronary artery diseases (CAD), such as angina and myocardial infarction (or a heart attack). Other CVDs may include stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

However, traditional cardiac monitoring and evaluation tools may not be well-suited to non-clinical environments. Equipment may be costly and difficult to use for untrained lay users. Cardiac monitoring equipment may involve numerous sensors, requiring specific placement, which may be difficult and time consuming for lay users to apply, and may be difficult for the lay users to apply to themselves, thereby preventing or discouraging regular use. Sensor cables can become tangled, pulled, and damaged, further frustrating the users and reducing equipment reliability. In addition, currently available cardiac monitors may provide continuous monitoring over a short period of time, such as 2 weeks or 30 days. This relatively short time period may be significant because cardiac conditions may manifest over a longer period of time, such as months or years. Thus, a short continuous monitoring window may not be useful for the lifetime of the disease.

The present disclosure provides methods and systems for determining a state or condition of a subject, such as a body part of the subject. Methods and systems of the present disclosure may be used to monitor a state or condition of an organ (e.g., a heart, lung, or bowel) or organ system (e.g., cardiovascular, pulmonary, gastrointestinal, or circulatory) of the subject, over various time periods. This may advantageously permit the subject to be monitored for a health or disease condition over a longer period of time.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1A shows a front perspective view of an exemplary monitoring device, in accordance with some embodiments.

FIG. 1B shows a back perspective view of the exemplary monitoring device of FIG. 1A, in accordance with some embodiments.

FIG. 2 shows a monitoring device comprising a stethoscope.

FIG. 3 shows schematic of a monitoring device placed external to a skin of a subject, in accordance with some embodiments.

FIG. 4 shows a schematic of a sensor unit in the interior of a monitoring device.

FIG. 5 shows a schematic of an interior of a monitoring device.

FIG. 6 shows an example packet structure for transmitting electrocardiogram (ECG) and audio data.

FIG. 7 shows a computer control system that is programmed or otherwise configured to implement methods provided herein.

FIG. 8A schematically shows a first exemplary electrode configuration of a monitoring device, in accordance with some embodiments.

FIG. 8B shows a wiring diagram of the first exemplary electrode configuration schematically shown in FIG. 8A while operating in an intrathoracic impedance measurement mode.

FIG. 8C shows the wiring diagram of the first exemplary electrode configuration schematically shown in FIG. 8A while operating in an ECG measurement mode.

FIG. 9 schematically shows a second exemplary electrode configuration of a monitoring device, in accordance with some embodiments.

FIG. 10 schematically shows example axes that may be used to determine an ECG vector.

FIG. 11A shows a QRS complex from an example ECG diagram.

FIG. 11B shows an audio waveform of an example heartbeat with time 0 being an R-peak of a QRS complex from an ECG recorded from the same heartbeat.

FIG. 12 shows examples of various heart murmurs with various shapes.

FIG. 13 shows an example of the interaction between different modules of a system, in accordance with some embodiments.

FIG. 14 shows a flow chart of an example method for utilizing an accelerometer in a monitoring device to gate an analysis of physiological data measured by the measuring device.

FIG. 15 shows a flow chart of an example method for utilizing an accelerometer in a monitoring device to adjust an audio gain of audio data transmitted from the measuring device to a listening device.

### DETAILED DESCRIPTION

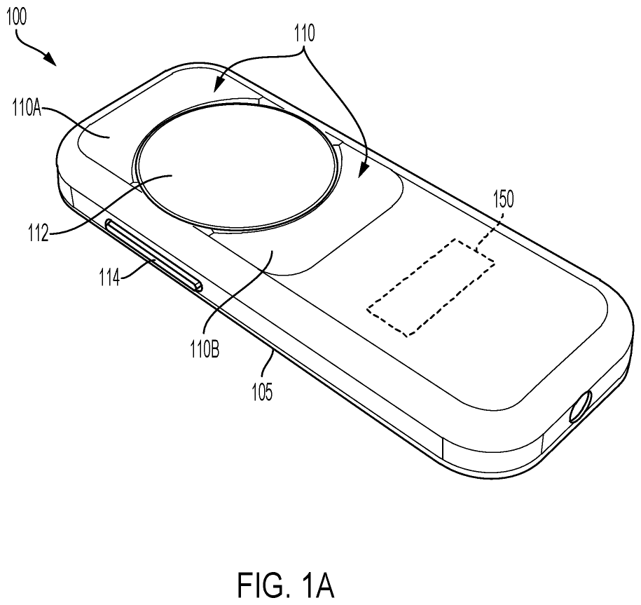

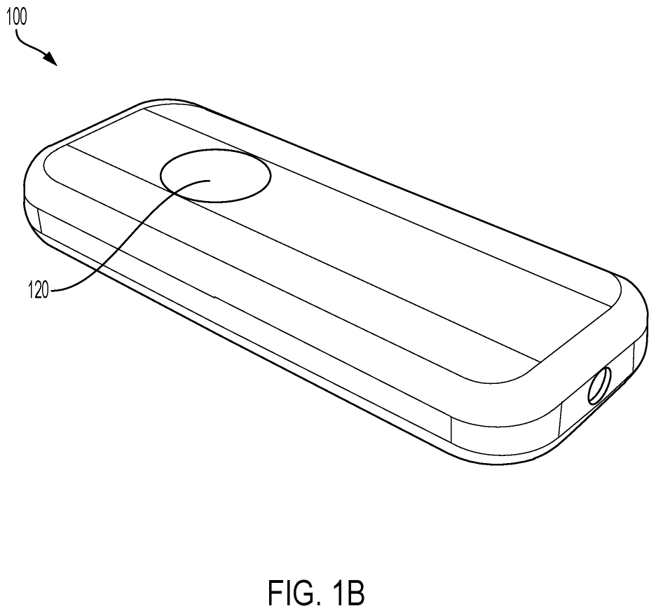

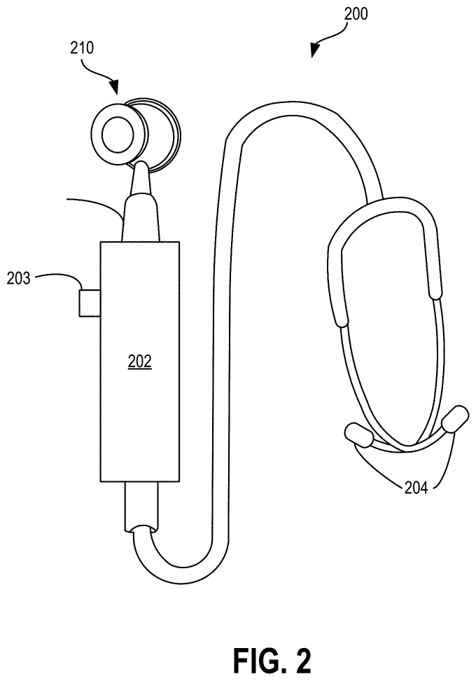

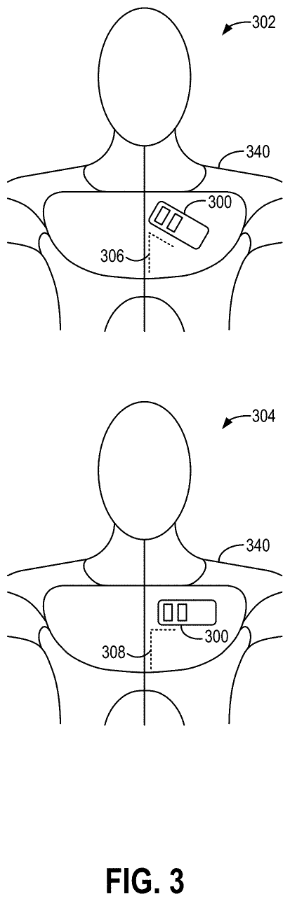

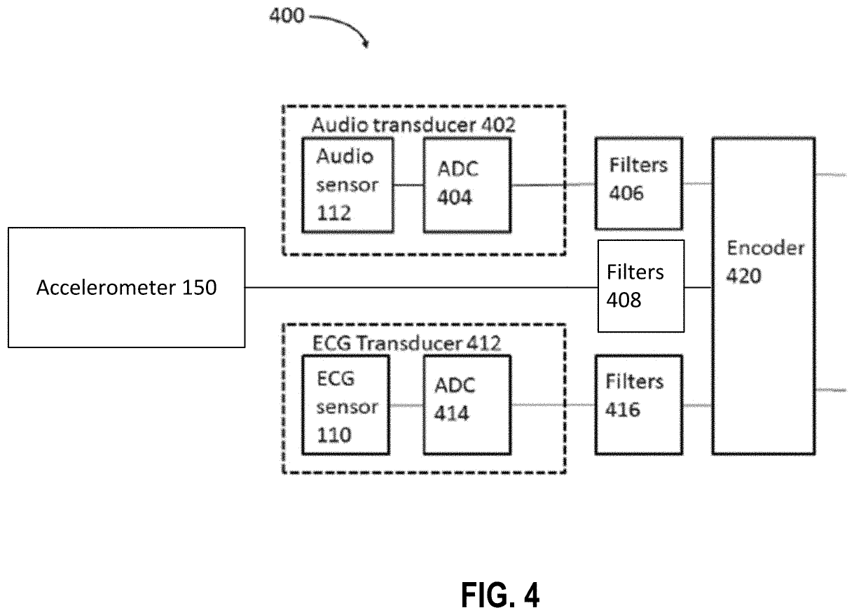

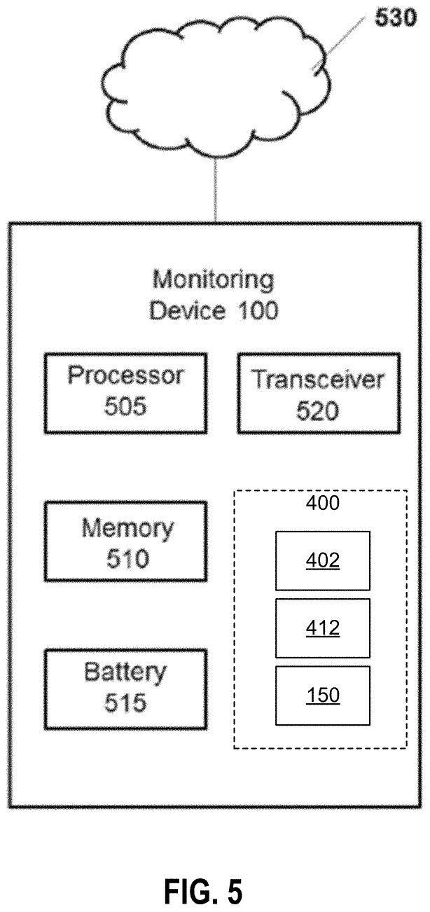

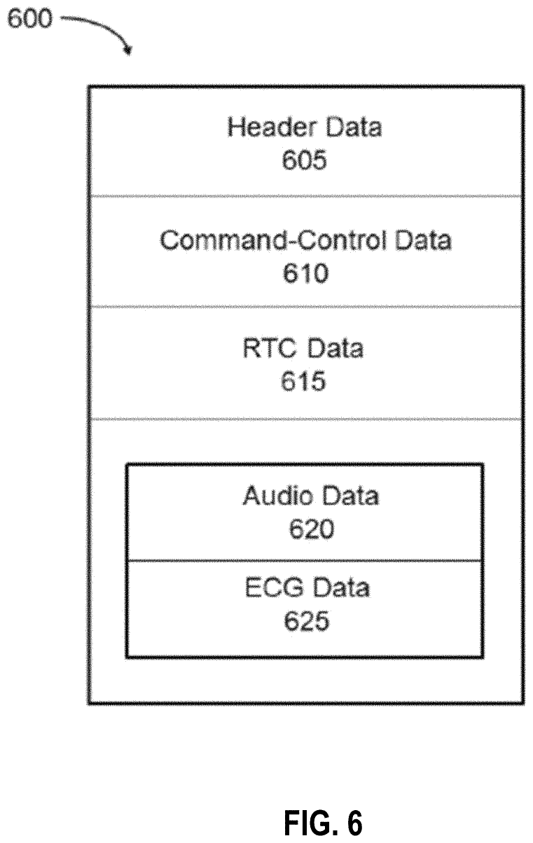

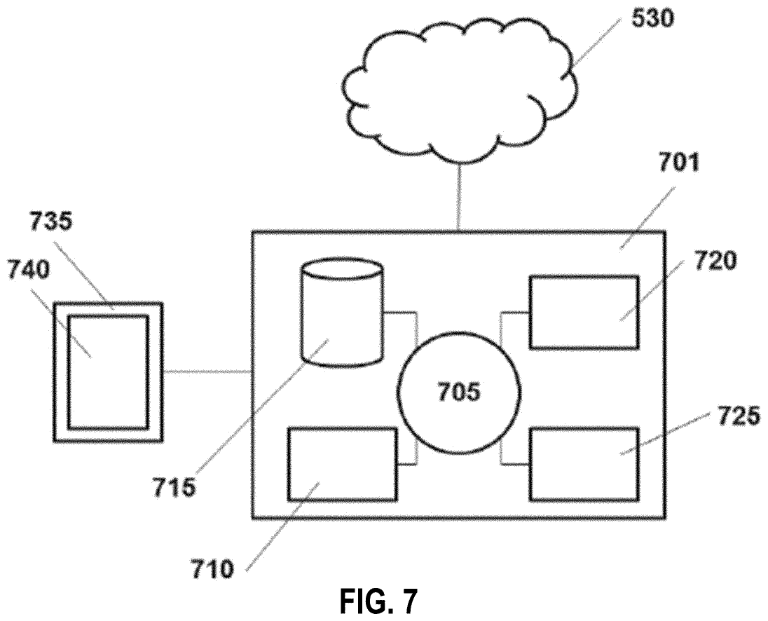

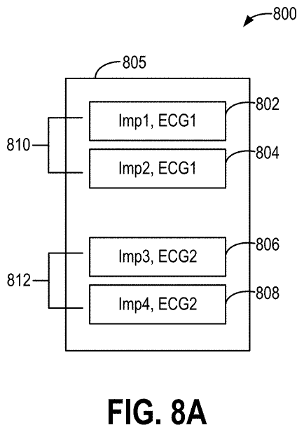

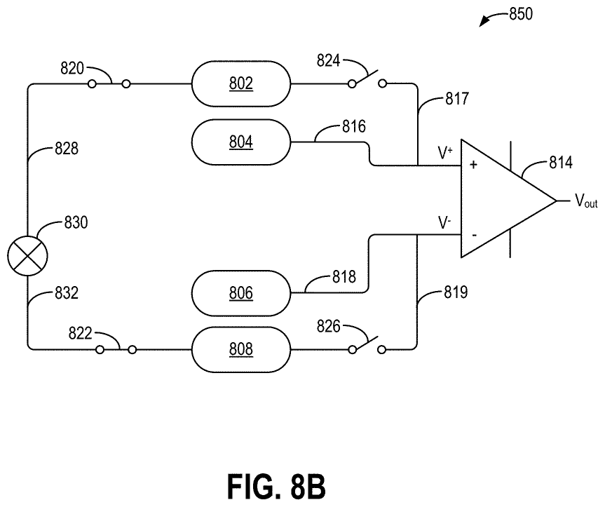

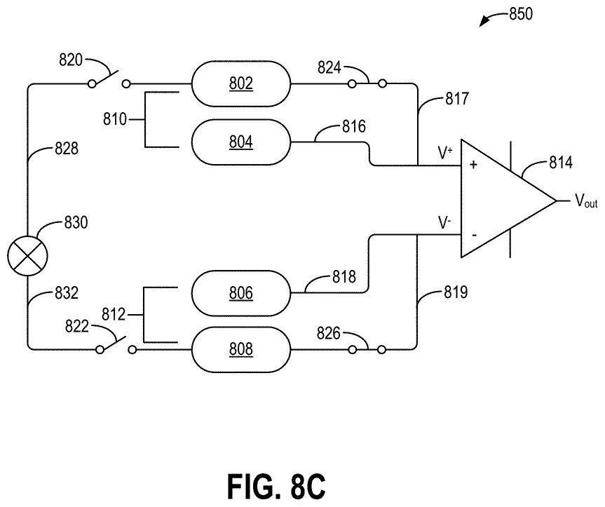

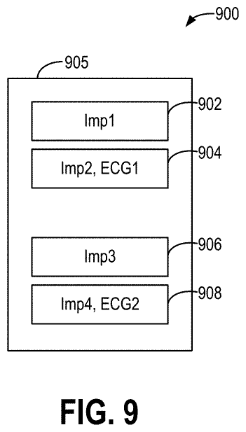

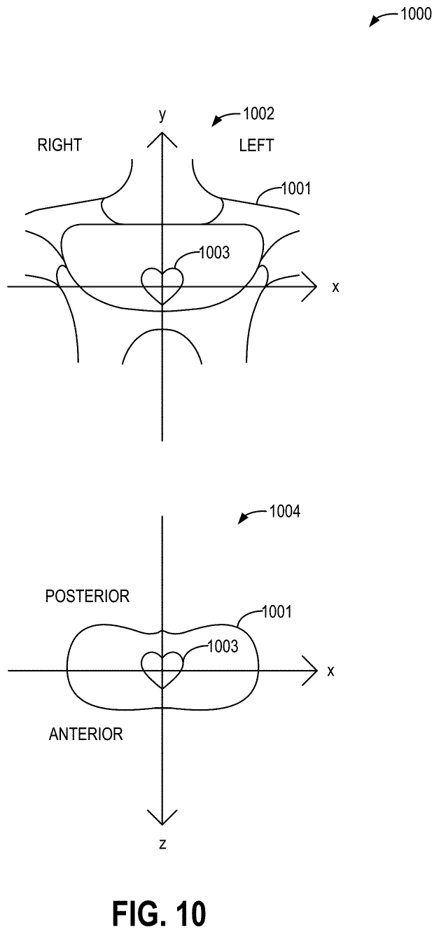

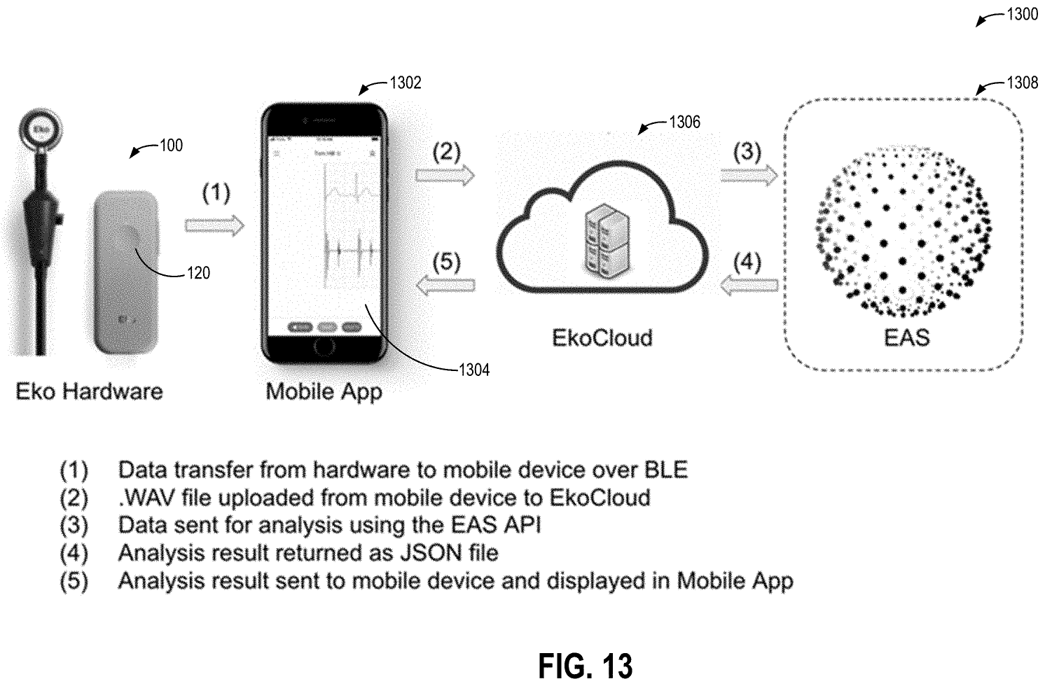

The following description relates to systems and methods for a digital health monitoring device, such as the monitoring device shown in FIGS. 1A and 1B. In some examples, the monitoring device may be a digital (e.g., electronic) stethoscope, such as shown in FIG. 2. As shown in FIG. 3, the monitoring device may be placed on a subject (e.g., patient), such as on a skin of the subject, in order to measure physiological data from the subject. The physiological data may include ECG data, intrathoracic impedance data, and/or audio data, for example. The monitoring device may include a sensor unit, such as the sensor unit shown in FIG. 4, that includes a plurality of sensor modalities for measuring the physiological data. In some examples, electrical sensors of the monitoring device may be used by both ECG and intrathoracic impedance sensor modalities, such as shown in FIGS. 8A-9. Further, the sensor unit may include an accelerometer, which may be used to determine an orientation of the monitoring device. The orientation of the monitoring device provides information regarding a vector of the measured ECG data with respect to example ECG vector axes schematically shown in FIG. 10. The monitoring device may include a processor and may be connected to a network, such as shown in FIG. 5. For example, the monitoring device may wirelessly transmit the physiological data using the example data packet structure shown in FIG. 6. Further, a computer system may receive the physiological data transmitted by the monitoring device, such as the computer system shown in FIG. 7, and may perform further processing and analysis of the physiological data. In some examples, the computer system may utilize cloud-based processing algorithms, such as diagrammed in FIG. 13.

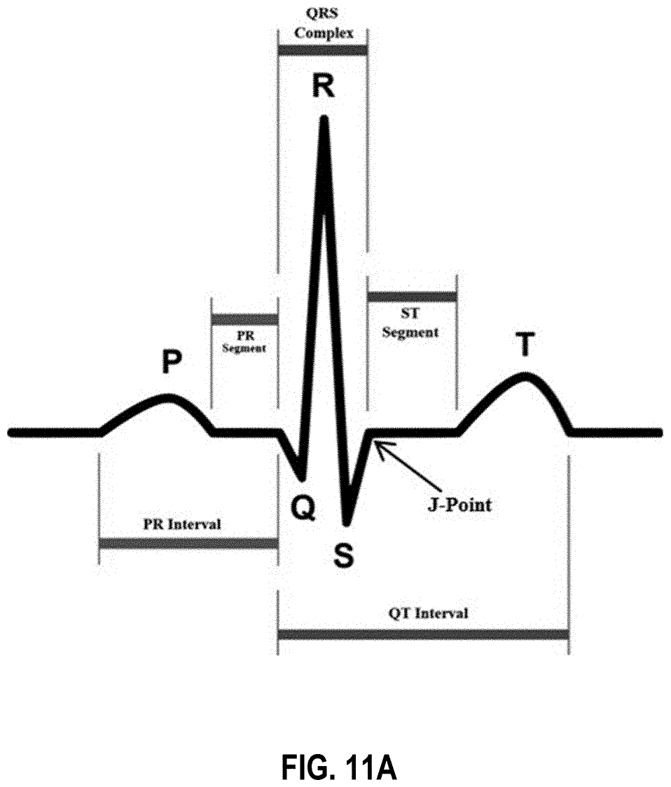

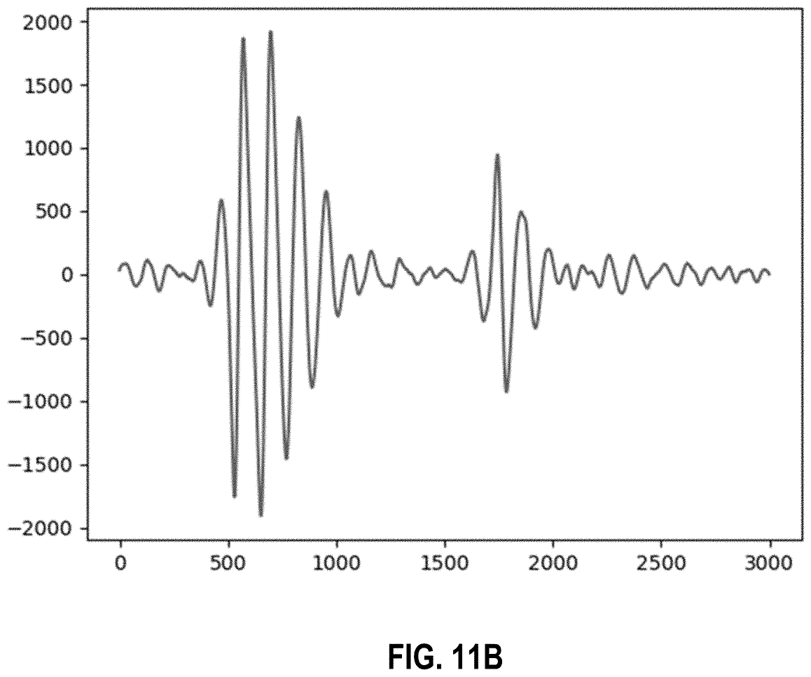

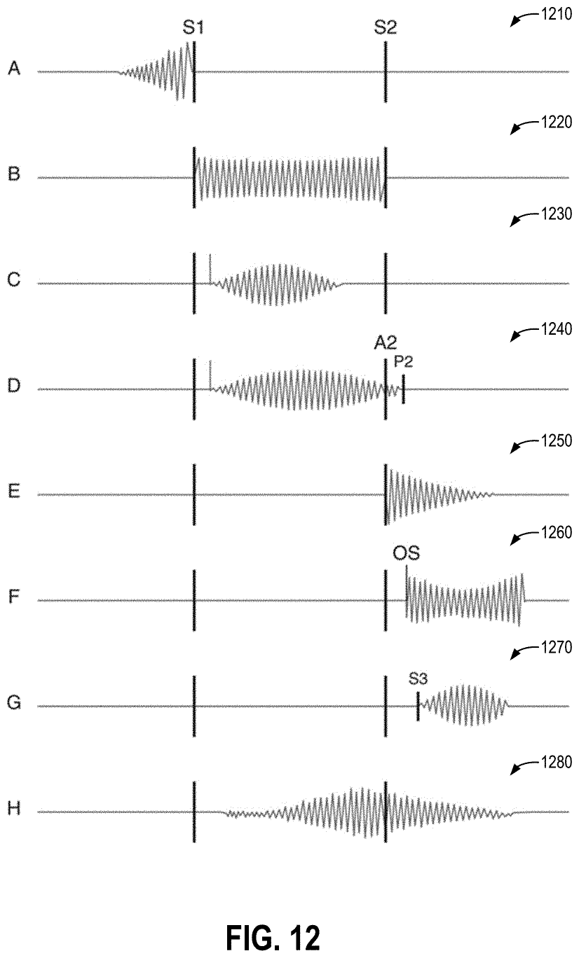

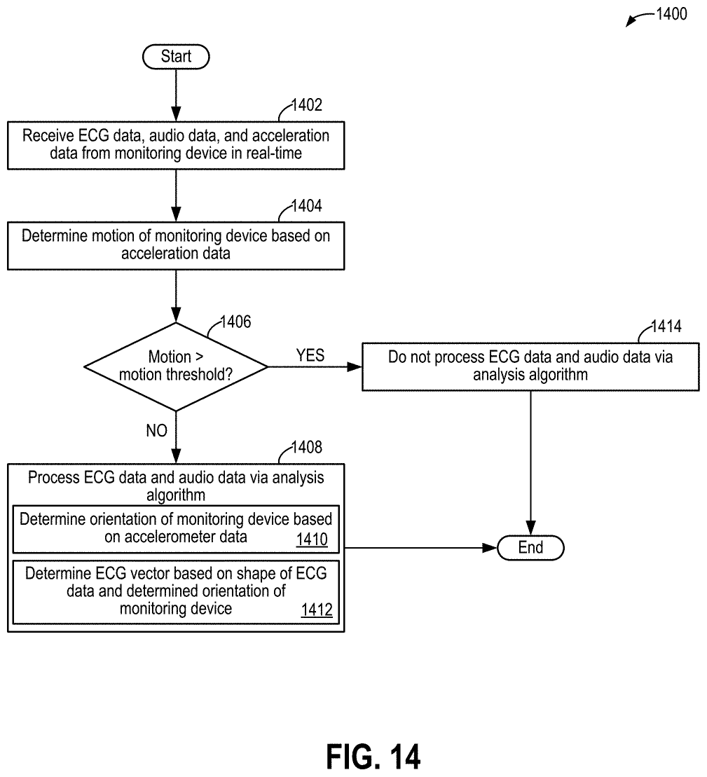

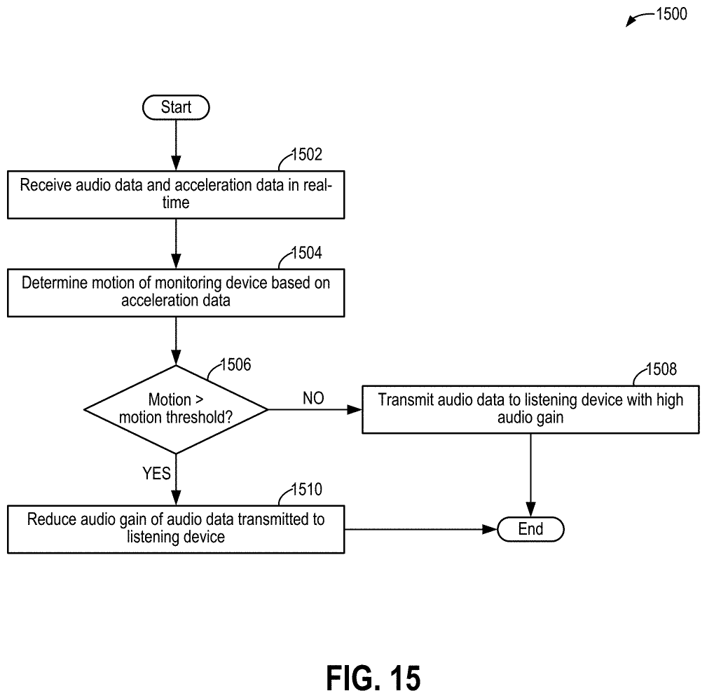

The processing and analysis of the physiological data may include constructing and analyzing ECG waveforms from the ECG data, such as the example ECG waveform shown in FIG. 11A, and heartbeat analysis from the audio data, such as the example audio waveform shown in FIG. 11B. Further still, the processing and analysis of the physiological data may include determining a state or condition of the subject, such as identifying different types of heart murmurs from the audio data. Examples of different audio waveforms representing different types of heart murmurs are shown in FIG. 12. The processing and analysis of the physiological data may further include using data from the accelerometer to gate processing of the ECG and audio data such that data obtained while the monitoring device is in motion is not used, such as shown in the example method of FIG. 14. In this way, the physiological data may be efficiently processed while increasing an accuracy of the resulting analysis and the determined state or condition of the subject. Further, a gain of audio data obtained while the monitoring device is in motion and transmitted to a listening device may be reduced, such as according to the example method of FIG. 15.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Unless otherwise defined, all technical terms used herein have the same meaning as commonly understood to one of ordinary skill in the art to which this disclosure belongs. As used in this specification and the appended claims, the singular forms “a,” “an,” and “the” include plural references unless the context clearly dictates otherwise. Any references to “or” herein is intended to compass “and/or” unless otherwise stated.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “monitoring device,” as used herein, generally refers to a device which comprises one or more sensors. In some examples, the one or more sensors may be coupled with or otherwise configured to be used in combination with the monitoring device. A sensor may be selected from various sensing modalities. The sensor may be capable of measuring sensor data over time. The monitoring device may include multiple sensors of the same type, such as multiple electrocardiogram (ECG) sensors or audio sensors. As an alternative, the monitoring device may include multiple sensors of different types, such as two or more sensors selected from an ECG sensor, audio sensor, temperature sensor, pressure sensor, vibration sensor, force sensor, respiratory monitor or sensor (e.g., a device, device part, or sensor capable of measuring a respiration rate of a subject), heart rate monitor or sensor, intrathoracic impedance monitor or sensor (e.g., a device, device part, or sensor capable of measuring an intrathoracic impedance), and/or other types of sensor, such as an accelerometer.

The monitoring device may be operable to connect to a remote device, such as a computing device. The monitoring device may be separated from the computing device. As an alternative, the monitoring device may be integrated with the computing device (e.g., the monitoring device and computing device may be components of the same device). In some examples, the monitoring device and the computing device may be the same device. The computing device may be a mobile device. The computing device may be separate from or external to the monitoring device. The monitoring device may be operable to connect to a remote server. Analysis of data measured from the monitoring device may be done on the monitoring device or on a separate computing device (e.g., a mobile device and/or a server).

The term “state or condition,” as used herein, generally refers to any classification which may be assigned to a subject or a part of a subject. The state or condition may comprise a disease state or healthy state. The state or condition may comprise a biological or physiological state or condition. The state or condition may comprise a particular diagnosis or determination. The state or condition may comprise an unknown state. Determining the state or condition of the subject may comprise determining the state or condition of an organ of the subject, such as, for example, a heart, lung, bowel, or other organ of the subject. For example, determining the state or condition of a heart may comprise a diagnosis or determination of a heart disease, disorder, or other condition such as low ejection fraction, normal ejection fraction, congestive heart failure, a heart failure risk score, heart murmur, innocent heart murmur, still's heart murmur, flow murmur, holosystolic or pansystolic murmurs, valve disease, arrhythmia (e.g., bradycardia, tachycardia, ventricular tachycardia, ventricular fibrillation, ventricular septal defect, premature ventricular contractions, patent ductus arteriosus, supraventricular arrhythmia, superventricular tachycardia (SVT), paroxysmal superventricular tachycardia (PSVT), atrial fibrillation, Wolff-Parkinson-White Syndrome, atrial flutter, premature supraventricular contractions or premature atrial contractions (PACs), postural orthostatic tachycardia syndrome (POTS)), congenital heart disease, heart blockage, ischemia, infarction, pericarditis, hypertrophy, etc. The diagnosis or determination of a heart murmur can comprise a diagnosis or determination of a systolic murmur or a diastolic murmur. Further, systolic murmurs may comprise an aortic stenosis, a pulmonic stenosis, a mitral regurgitation, a tricuspid regurgitation, or a mitral valve prolapse, and more. Diastolic murmurs may comprise an aortic regurgitation, a pulmonic regurgitation, a mitral stenosis, or a tricuspid stenosis, and more. For example, determining the state or condition of a lung may comprise a diagnosis or determination of a lung disease, disorder, or other condition such as pneumonia, plural effusion, pulmonary embolism, poor airflow, chronic obstructive pulmonary disease (COPD), asthma, etc. For example, determining the state of condition of a bowel may comprise a diagnosis or determination of a bowel disease, disorder, or other condition such as inflammatory bowel disease (IBD), intestinal obstruction, hernia, infection within the digestive tract, or other condition, such as any condition disclosed elsewhere herein.

The term “subject,” as used herein, generally refers to an animal, such as a mammal (e.g., human) or avian (e.g., bird), or other organism. A subject can be a healthy or asymptomatic individual, an individual that has or is suspected of having a disease (e.g., cancer) or a pre-disposition to the disease, and/or an individual that is in need of therapy or suspected of needing therapy. The subject may be symptomatic with respect to a disease or condition. Alternatively, the subject may be asymptomatic with respect to the disease or condition. The subject can be a patient.

The term “algorithm,” as used herein, generally refers to a process or rule for conducting a calculation or other problem-solving operation. An algorithm may be implemented by a computer, which may comprise one or more computer processors or circuitry for executing the algorithm, as described elsewhere herein. The algorithm may be a trained algorithm. The algorithm may be trained with one or more training sets, which may include data generated from subjects with known physiological or biological states or conditions. The trained algorithm may comprise a machine learning algorithm, such as a supervised machine learning algorithm or an unsupervised machine learning algorithm.

The term “real-time,” as used herein, can refer to a response time of less than or equal to about 1 second, a tenth of a second, a hundredth of a second, a millisecond, or less. The response time may be greater than about 1 second. In some instances, real-time can refer to simultaneous or substantially simultaneous processing, detection, or identification. As another example, the term “real-time” refers to a process executed without intentional delay.

### Monitoring Devices

The present disclosure provides monitoring devices that may be used to collect data indicative of a physiological or biological state or condition of a subject, such as an organ or an organ system of the subject. In some examples, the organ systems may comprise a subject's cardiovascular, respiratory, digestive system, and/or other organ systems or body parts. Organs may comprise heart, lung, bowel, skin, or other body parts and/or organs of the subject.

In some examples, the monitoring device may comprise or be a hand-held device, such as a device which may be configured to be held in the hand of a user. The monitoring device may be configured to be placed upon a body part of the subject, such as, for a duration of time, for example, during the monitoring. The device may be placed upon any body part for monitoring purposes. Such body parts may comprise chest, abdomen, back, neck, head, skin, or any other parts of the body. In some examples, the monitoring device may be hand-held. Alternatively, the monitoring device may not be hand-held. The monitoring devices may be configured for use with computing devices described elsewhere herein. Further, the monitoring device may be configured to comprise a computing device.

In some examples, the monitoring device may be configured to be in the form of a patch or an adhesive pad, that is configured to attach or adhere to a skin of a patient. The monitoring device may remain attached to the skin of the subject for a duration of time, for example, for the duration of the monitoring. The patches may be re-usable. Alternatively, the patches and/or pads may be single-use or disposable. The patches may be made of any suitable material. In some examples, the patches or pads may comprise or be used in combination with a polymeric material and gel. The gel may comprise potassium chloride, silver chloride, and/or other material. The gel may permit, facilitate, or enhance electron conduction from the skin to the monitoring device (e.g., through a wire).

FIG. 1A shows a top view of a monitoring device 100 comprising a housing 105, which encases sensors and control circuitry. The monitoring device 100 comprises an electrical sensor 110 of a first sensor modality and an audio sensor 112 of a second sensor modality positioned on an exterior of the housing 105. In the illustrated example, the electrical sensor 110 includes a first electrode 110A and a second electrode 110B, however, other numbers of electrodes are possible, such as will be described with respect to FIGS. 8 and 9. The first electrode 110A and a second electrode 110B may comprise contact pads of an ECG sensor, for example. Additionally or alternatively, the first electrode 110A and the second electrode 110B may comprise a current injection electrode and a voltage measurement electrode, respectively, for intrathoracic impedance measurements. For example, as will be elaborated herein with respect to FIGS. 8 and 9, each of the first electrode 110A and the second electrode 110B may be used to measure both ECG and intrathoracic impedance. The audio sensor 112 may comprise a surface for obtaining audio data. The audio sensor 112 may include one or more microphones units for collecting audio data. It may be understood that additional sensor modalities may be positioned internal to the housing 105, such as a third sensor 150 schematically indicated by a dashed box. In one example, third sensor 150 is an accelerometer.

The monitoring device 100 may additionally comprise user controls such as a button 114. The button 114 may control the intensity of a monitored signal to be transmitted to a user. The button 114 may comprise a positive end and a negative end, such that when the positive end (e.g., a first end) of the button is depressed, a signal amplitude is increased, and when a negative end (e.g., a second end opposite the first end) of the button is depressed, the signal amplitude is decreased. The signal amplitude may comprise a volume of an amplified audio signal. The audio signal may be transmitted wirelessly to an earpiece of a user (such as a healthcare provider) or of a subject.

FIG. 1B shows a bottom view of the monitoring device 100. The monitoring device 100 may comprise additional user controls such as a button 120. The button 120 may be used to stop and start measurement of data by the monitoring device 100. The button 120 may be actuated by a user. It may be possible to stop or start measurement without actuating the button 120, such as by controlling collection through a computing device. The shape and design of the housing 105 may facilitate a subject's comfort during monitoring a state or condition of the subject. Additionally, the shape and design of the housing 105 may facilitate a secure fit against a variety of patient body types and shapes in order to increase sensor contact and with adequate sensor geometry.

The monitoring device 100 may comprise one or more sensors. In some examples, the monitoring device 100 comprises at least three sensors (e.g., sensor modalities). The monitoring device 100 may comprise 1, 2, 3, 4, 5, 6, 7, 8, 9, 10, or more sensors of the same or of different types. The sensors may be various types of sensors, such as ECG sensors, audio sensors, temperature sensors, pressure sensors, vibration sensors, force sensors, respiratory monitors or sensors (e.g., a device, device part, or sensor capable of measuring a respiration rate), heart rate monitors or sensors, intrathoracic impedance monitors or sensors (e.g., a device, device part, or sensor capable of measuring an intrathoracic impedance), accelerometers, and/or other types of sensors. The sensors may be part of the monitoring device 100. In other examples, the sensors may be coupled with or otherwise configured to be used in combination with the monitoring device 100 and/or the computing device. The one or more sensors may be sensors of any kind, shape, form, or material.

The one or more sensors may comprise one or more vibration and/or force sensors. The one or more vibration or force sensors may be used to measure a force in an organ or organ system of the subject. For example, the vibration or force may be configured to perform cardiac force measurement. The force sensor may be a cutaneous sensor, a precordial cutaneous sensor, a piezoelectric cantilever sensor, a transcutaneous force sensor, or other type of sensor. In some examples, a vibration or force sensor (e.g., a pressure sensor) may be configured to measure a pressure, a force, or a vibration in a body part such as heart or lung. For example, a systolic pressure may be measured from the heart of the subject. In an example, the force can be measured as the myocardial vibrations amplitude in an isovolumic contraction period.

Data from vibration, force, or pressure sensors may be used, individually or in combination with data received from the other sensors of the monitoring device 100, to detect or identify a state or condition of the subject, such as a pressure (e.g., an increased pressure or filling pressure) inside the heart, such as pulmonary artery pressure, pulmonary arterial wedge pressure, central venous pressure, jugular venous pressure, left ventricle end-diastolic pressure (LVEDP), or other kinds of pressure. The states or conditions that can be detected using a vibration or force sensor, individually or in combination with other sensors of the monitoring device 100 may further comprise conditions such as a contraction. A contraction may be present, for example, in the heart of the subject. A contraction may comprise a ventricular contraction (e.g., a premature ventricular contraction), an atrial contraction (e.g., a premature atrial contraction), or other types of contraction. As an example, a ventricular myocardium may produce a force of contraction which may increase in response to an increase in a contraction frequency. This may be referred to as a cardiac force-frequency relation (FFR). A force can be measured and/or computed as the systolic pressure/end-systolic index ratio, which may be measured, for example, for increasing heart rates, for example, when the subject is under stress (e.g., emotional stress). In some examples, data measured using a force, vibration, or pressure sensor can be used to build a curve of force variation as a function of heart rate.

The accelerometer may comprise a three-axis accelerometer, which may provide information about the orientation and motion of the monitoring device 100. The accelerometer may be rigidly affixed to a surface within the monitoring device 100 so that the accelerometer does not move independently from the monitoring device 100 as a whole. The accelerometer may be used to calculate an orientation of the monitoring device 100 when the monitoring device 100 is held stationary by a user, such as the subject or a healthcare professional. As will be elaborated herein with respect to FIGS. 3 and 10, the orientation of the monitoring device 100 may be used by an algorithm in combination with a shape of ECG data (e.g., as recorded by the electrical sensor 110) to predict an ECG vector being measured. Further, the motion of the monitoring device 100 measured by the accelerometer may be used to gate processing of the EGC data and/or audio data, as will also be described herein with respect to FIG. 14.

The one or more sensors may comprise a sensor for measuring a respiration rate of the subject. In some examples, the accelerometer (e.g., the third sensor 150) may be used to measure the respiration rate. The one or more sensors may comprise a sensor for measuring an intrathoracic impedance, such as the electrical sensor 110. Measuring intrathoracic impedance may provide information about a presence or the amount of a fluid in the lungs of the subject. For example, intrathoracic impedance may decrease as an amount of a fluid in the lung or lungs increases. The reason for this may be that the fluid may be a conductor of electrical current. Data collected using intrathoracic impedance sensors may provide insight and information about the condition of the lungs of the subject and identify potential signs of decompensation, pulmonary edema, or any state or condition of the subject, such as states or conditions correlated with the presence of fluid in lungs. For example, wheezes, crackles and rhonchi are often heard in lung sounds due to fluid accumulation in the lungs. Therefore, the intrathoracic impedance measurement may be used in conjunction with lung sounds obtained by the audio sensor 112 to provide a joint measure of fluid retention.

The monitoring device 100 may be mobile. For example, the monitoring device 100 may be capable of movement from one point to another. The monitoring device 100 may be capable of placement on and removal from a body of the subject. For example, the monitoring device 100 may be placed adjacent to the body of the subject at a location in proximity to a heart, lung, or bowel of the subject. The monitoring device 100 may not be implantable in the body of the subject. The monitoring device 100 may be sufficiently light that it is easily transported from one location to another. For example, the device may weigh no more than about 0.5 pounds, 1 pound, 2 pounds, 3 pounds, 4 pounds, 5 pounds, 6 pounds, 7 pounds, 8 pounds, 9 pounds, 10 pounds, or 50 pounds.

The monitoring device 100 may be used to collect ECG data, audio data, intrathoracic impedance data, and/or orientation and motion data from a plurality of different locations or parts of a body of the subject, such as positions at and/or around a heart, lung, vein, or artery of the subject. In some examples, the monitoring device 100 may further comprise more sensors, such as sensors listed anywhere herein which can be used to collect data from various parts of the subject's body. Data collection may be performed by placing the monitoring device 100 or the one or more sensors at different positions adjacent to the body of the subject (e.g., in contact with the body, inside the body, or remote from the body) and using the monitoring device 100 to take one or more measurements (e.g., collect ECG data, audio data, intrathoracic impedance data, orientation and motion data, or any other type of data) at each of at least a subset of the different positions at suitable time points and/or intervals for suitable durations of time.

In an example, the monitoring device 100 may be used to collect the audio data of the patient to evaluate the status of the lungs or heart. In some examples, the monitoring device 100 may be used to input a sound into the patient and record the sound reflection to indicate the status of a status of condition of the subject (e.g., body tissue or fluid levels).

The monitoring device 100 may be sufficiently sized such that it is easily transported from one location to another. The monitoring device 100 may be handheld. The monitoring device 100 may be of a size which may fit in a hand. For example, the monitoring device 100 may comprise an external dimension of less than or equal to about 0.25 inches, 0.5 inches, 1 inch, 2 inches, 3 inches, 4 inches, 5 inches, 6 inches, 7 inches, 8 inches, 9 inches, 10 inches, 11 inches, 12 inches, or 24 inches.

The monitoring device disclosed herein may be an electronic stethoscope 200, as illustrated in FIG. 2. The electronic stethoscope 200 includes a resonator 210 that is configured to be placed adjacent to a body of a subject. The resonator 210 may be disc shaped. The resonator 210 may be configured to collect audio information from a body of the subject, similar to the audio sensor 112 of FIGS. 1A and 1B. The resonator may include, for example, a piezoelectric unit and circuitry for collecting audio information from the body of the subject. The resonator 210 may include circuitry with a communication interface that may be in communication (wired or wireless communication) with a transmitting unit 202 that includes a button 203. The electronic stethoscope 200 may comprise earpieces 204. The earpieces 204 may be used to listen to audio data as they are being generated. The earpieces 204 may also be used to provide audio feedback generated by the trained algorithm to the user or the healthcare provider. Upon a user pressing the button 203, audio information may be collected from the body of the subject and stored in memory and/or transmitted to a mobile device (e.g., mobile computer) in communication with the transmitting unit 202. Further, the electronic stethoscope 200 may include all or some of the sensor modalities described above with respect to FIGS. 1A and 1B. For example, the electronic stethoscope 200 may be one embodiment of the monitoring device 100, and the resonator 210 may be one embodiment of the audio sensor 112.

FIG. 3 shows a monitoring device 300 placed external to a skin of a subject 340. The monitoring device 300 may be the monitoring device 100 of FIGS. 1A and 1B or the electronic stethoscope 200 of FIG. 2, for example. The position of the monitoring device 300 may be varied with respect to anatomical features of the subject 340 depending on the state or condition to be characterized. For example, the position of the monitoring device 300 may be external to the skin near the subject's heart. As another example, the position of the monitoring device may be near the subject's lung. As still another example, the position of the monitoring device 300 may be near the subject's bowel. In yet another example, the position of the monitoring device 300 may be near the subject's fistula (e.g., a diabetic fistula). The monitoring device 300 may be placed on the skin of the subject 340 upon or near one or more areas of the head, chest, foot, hand, knee, ankle, or other body part of the subject 340. The monitoring device 300 may be used to obtain indications for venous access, which is one of the most basic but useful components of patient care both in hospital, in dialysis clinics, and in ambulatory patient settings. The monitoring device 300 may be used to obtain indications of the flow rate or status of a fistula for venous access. The monitoring device 300 may be used to obtain indications of lung fluid status for heart failure patients. The monitoring device 300 may be used to obtain indications of cardiac filling pressure for heart failure patients. The monitoring device 300 may be used to obtain indications to prescribe or not prescribe a medication based upon an output of a QT interval of the subject 340. The monitoring device 300 may be used to obtain indications to change a medication dosage or frequency based upon the QT interval of the subject 340. The monitoring device 300 may be used to obtain indications to change a heart failure medication prescription, dosage, or frequency, such as a diuretic or ace inhibitor, based upon the cardiac output, systolic time intervals, or lung fluid status.

It may be beneficial to place the monitoring device 300 such that surface of a sensor, such as the electrical sensor 110 described above with respect to FIG. 1A, is substantially in contact with the subject's skin. The sensors may be substantially in contact when a majority of the sensor surface is in contact with the subject's skin. In some examples, pressure directed toward the skin may be applied onto the monitoring device 300 in order to increase the surface area of the sensors in contact with the skin. Pressure may be applied by the subject 340 or a third party (e.g., a healthcare professional). However, it may be possible to determine the state or condition of the subject 340 without applying pressure to the monitoring device 300. It may be beneficial to apply a conductive gel to increase electrical contact between the sensor and the skin. The conductive gel may be beneficial in examples where the subject has particularly dry skin or has significant body fat or hair, for example.

The orientation of the monitoring device 300 on the subject 340 may be adjusted by rotating the device relative to the surface of the skin of the subject 340. Two example orientations are shown in FIG. 3, including a first orientation 302 and a second orientation 304. For example, each orientation may be at least partially defined by an angle between a length of the monitoring device 300 with respect to a midline (or sternum) of the subject 340. The angle may be at least about 5, 10, 20, 30, 40, 45, 50, 60, 70, 80, 90, 100, 110, 120, 130, 140, 150, 160, 170, 180, 270, or greater degrees relative to the sternum, or any angle within by a range defined by any two of the preceding values. The first orientation 302 includes a first angle 306, and the second orientation includes a second angle 308, which is wider than the first angle 306. In the example shown, the first angle 306 is an acute angle, while the second angle 308 is a right angle. In other examples, the monitoring device 300 may be placed at an obtuse angle. Thus, the first orientation 302 and the second orientation 304 include the monitoring device 300 at a same general position of the subject 340 (e.g., on the upper chest near the heart) but at different rotational orientations. The different orientations may produce different ECG vectors because of the different positions of the ECG electrodes (e.g., the first electrode 110A and the second electrode 110B shown in FIG. 1A) with respect to depolarization and repolarization of muscle cells in the heart.

Referring now to FIG. 10, ECG vector axes 1000 are schematically shown across two views. The ECG vector axes 1000 include three mutually perpendicular axes of a Cartesian coordinate system centered on a heart 1003 of a subject 1001. A first view 1002 shows a y-axis increasing in a cranial direction from the heart 1003 and an x-axis increasing leftward from the heart 1003. A second view 1004 shows the x-axis and additionally shows a z-axis increasing in an anterior direction from the heart 1003. When ECG leads are positioned in different locations with respect to the x-, y-, and z-axes, different ECG vectors may be obtained, which may affect the resulting waveform and analysis. Although the example shown in FIG. 10 includes three mutually perpendicular ECG vector axes of a Cartesian coordinate system, other vector axes and coordinate systems may be used in determining the ECG vector.

Returning to FIG. 3, the monitoring device 300 may include an accelerometer, such as the accelerometer 150 shown in FIG. 1A. Output from the accelerometer may be used to calculate the orientation of the monitoring device 300 when the monitoring device is held stationary, such as the angle of the monitoring device 300. However, the accelerometer may not measure the position of the monitoring device 300 on the subject 340. Therefore, the accelerometer alone may not provide enough information to determine the ECG vector being measured. However, because a shape of the ECG waveform changes based on the ECG vector being measured, the orientation of the monitoring device 300 measured by the accelerometer in combination with the shape of the recorded ECG may be used by an algorithm to predict the ECG vector being measured, as will be elaborated below with respect to FIG. 14. Thus, knowledge of the orientation of the monitoring device 300, determined from the output of the accelerometer, may be used in determining which ECG vector is measured by the ECG sensor.

### Sensor Modalities

The monitoring device described herein (e.g., the monitoring device 100 shown in FIGS. 1A and 1B) may comprise sensors of one or a plurality of sensor modalities. The modalities may be operable to measure data from a subject. Examples of sensor modalities include electrical sensors (e.g., conductivity sensor, charge sensor, resistivity sensor, or impedance sensor), audio sensors, accelerometers, light sensors, etc. The sensors may comprise ECG sensors, audio sensors, temperature sensors, pressure sensors, vibration sensors, force sensors, respiratory monitors or sensors (e.g., a device, device part, or sensor capable of measuring a respiration rate), heart rate monitors or sensors, intrathoracic impedance monitors or sensors (e.g., a device, device part, or sensor capable of measuring an intrathoracic impedance), and/or other types of sensors. The ECG sensor and the audio sensor may record ECG and audio data. The monitoring device may comprise at least two sensor modalities. Additionally or alternatively, the monitoring device may comprise at least three, at least 4, at least 5, or more sensor modalities. The monitoring device may comprise a plurality of sensors of each sensor modality. For example, the monitoring device may comprise at least about 1, 2, 3, 4, 5, 10, 20, 30, 40, 50, 100, 200, 300, 400, 500, or more sensors of any individual sensor modality. The number of sensors of a single modality may be the same; alternatively, there may be more or fewer sensors of one modality than another.

The monitoring device may include a housing having at least one ECG sensor and at least one audio sensor. The at least one ECG sensor may be integrated with the at least one audio sensor. The monitoring device may include at least about 1, 2, 3, 4, 5 or more ECG sensors, and at least about 1, 2, 3, 4, 5 or more audio sensors. The housing may further comprise other sensors of any type, such as any sensor listed anywhere herein at any number. For example, the housing may have at least 0, 1, 2, 3, 4, 5, 6, 7, 8, 10, 12, 15, 20, or more of each sensor.

In some examples, the device comprises a plurality of sensor modalities, such as at least 1, 2, 3, 4, 5, 10, 20, 30, 40, 50, or more sensor modalities which may be referred to as the first sensor modality, the second sensor modality, and so on, respectively. The first sensor modality may be an electrical sensor. Referring to FIGS. 1A and 1B, the electrical sensor 110 may comprise the first electrode 110A and the second electrode 110B. The first electrode 110A and the second electrode 110B may be physically separated by a distance to facilitate measurement of an electrical signal from a subject. The distance between the first and second electrodes may be at least about 1 millimeter (mm), 2 mm, 5 mm, 10 mm, 20 mm, 50 mm, 100 mm, 200 mm, 500 mm, or more, or any distance defined by a range between any two of the preceding values. The first electrode 110A and the second electrode 110B may comprise ECG transducer electrodes, which may measure electrical signals from a subject resulting from depolarization of the heart muscle during a heartbeat.

In some examples, the data (e.g., ECG data, audio data and/or any other type of data, such as data listed anywhere herein) may be measured from the organ (e.g., heart, lung, or bowel) of the subject over a time period. Such time period may be at least about 5 seconds, 10 seconds, 20 seconds, 30 seconds, 1 minute, 5 minutes, 10 minutes, 30 minutes, 1 hour, 2 hours, 3 hours, 4 hours, 5 hours, 6 hours, 12 hours, 1 day, or more. Alternatively, such time period may be at most about 6 months, 3 months, 2 months, 1 months, 3 weeks, 2 weeks 1 week 6 days, 5 days, 4 days, 3 days, 1 day, 12 hours, 6 hours, 5 hours, 4 hours, 3 hours, 2 hours, 1 hour, 30 minutes, 10 minutes, 5 minutes, 1 minute, 30 seconds, 20 seconds, 10 seconds, 5 seconds, or less. The time period may be from about 1 second to 5 minutes, from about 5 seconds to 2 minutes, from about 10 seconds to 1 minute, from about 1 minute to 10 minutes, from about 10 minutes to 1 hour, or from about 1 minute to 1 hour.

In some examples, the data (e.g., ECG data and audio data and/or any other type of data, such as data listed anywhere herein) is measured from the organ (e.g., heart, lung, or bowel) of the subject over multiple time periods. The one or more time periods may be discontinuous. The one or more time periods may be temporally separate from other time periods. For example, the ECG and audio data may be measured over a first time period, the ECG data and audio data may be not be measured for an intervening period, and the ECG data and audio data may be measured over a second time period. The intervening period may be at least about 1 minute, 5 minutes, 10 minutes, 1 hour, 5 hours, 10 hours, 1 day, 1 week, 1 month, 1 year, 5 years, 10 years, or more. The intervening period may be from about 1 minute to 10 minutes, from about 1 minute to 1 hour, from about 5 minutes to 5 hours, from about 1 hour to 1 day, from about 1 day to 1 week, from about 1 week to 1 month, or from about 1 week to 1 year. The same can apply to other data collected using the monitoring device (e.g., using the one or more sensors, any of the sensor modalities, or any combination thereof).

In some examples, many time periods may be separated by many intervening periods. In such an example, a longitudinal dataset comprising subject data may be collected. A longitudinal data set may be used to track a state or condition of a subject (such as the state or condition of a heart of a subject) over an extended period of time. A monitoring device may track an output comprising a state or condition of a subject over time. Additionally, a monitoring device may track a diagnostic feature of a subject over time. In some examples, ECG data from a first period may be compared to ECG data from a second period. In some examples, audio data from a first period may be compared to audio data from a second period. In some examples, combined datasets or features based on combined datasets may be compared. Such datasets may comprise any combination of data and/or datasets collected using any combination of sensors and/or sensor modalities.

A device of the present disclosure may include at least 1, 2, 3, 4, 5, 6, 7, 8, 9, 10, or more ECG electrodes. In an example, the monitoring device may comprise one or more electrodes (e.g., ECG electrodes) which may be placed at certain angles relative to one another to create one or more (e.g., multiple) ECG vectors. The one or more electrodes may comprise at least 1, 2, 3, 4, 5, 6, 7, 8, 9, 10, or more electrodes. In some examples, the electrodes may be placed at orthogonal angles to create multiple orthogonal ECG vectors. As an alternative or in addition to, the device may include at least 1, 2, 3, 4, 5, 6, 7, 8, 9, 10, or more audio sensors.

Turning now to FIG. 8A, a first exemplary electrode configuration 800 of a monitoring device is shown. The monitoring device may be the monitoring device 100 of FIGS. 1A and 1B, for example. The first exemplary electrode configuration 800 includes a first electrode 802, a second electrode 804, a third electrode 806, and a fourth electrode 808 positioned in a housing 805. In the example shown in FIG. 8A, each of the four electrodes is shared by an ECG sensor and an intrathoracic impedance sensor. Thus, each of the four electrodes is used by two sensor modalities. In the present example, the first electrode 802 is a first impedance electrode (Imp1) used for current injection, the second electrode 804 is a second impedance electrode (Imp2) used for voltage measurement, the third electrode 806 is a third impedance electrode (Imp3) used for voltage measurement, and the fourth electrode 808 is a fourth impedance electrode (Imp4) used for current injection. For example, the first electrode 802 and the fourth electrode 808 are configured to inject a high frequency, low amplitude current into a subject, and the second electrode 804 and the third electrode 806 are configured to sense the resulting electrical potential from the injected current. Because four intrathoracic impedance electrodes are used (e.g., two to inject current and two to sense voltage), skin-electrode impedance will not be included in the voltage measurement. Further, the first electrode 802 and the second electrode 804 are functionally coupled together to form a first ECG electrode 810 (ECG1), while the third electrode 806 and the fourth electrode 808 are functionally coupled together to form a second EGC electrode 812 (ECG2).

Referring now to FIGS. 8B and 8C, an example sensor circuit 850 for the first exemplary electrode configuration 800 is shown. The sensor circuit 850 includes the second electrode 804 and the third electrode 806 electrically coupled to a differential amplifier 814. The differential amplifier 814 outputs a voltage, Vₒᵤₜ, that is proportional to a difference between a first voltage input V⁺ and a second voltage input V⁻. For example, a first wire 816 electrically coupled to the second electrode 804 provides the first voltage input V⁺ to the differential amplifier 814, and a second wire 818 electrically coupled to the third electrode 806 provides the second voltage input V⁻ to the differential amplifier 814. The first electrode 802 and the fourth electrode 804 are electrically coupled to either the differential amplifier 814 or a current source 830 based on a position of each of a plurality of switches, as elaborated below.

While operating the sensor circuit 850 in an intrathoracic impedance measurement mode shown in FIG. 8B, the first electrode 802 and the fourth electrode 808 are not connected to the differential amplifier 814. Instead, the first electrode 802 and the fourth electrode 808 are electrically coupled to the current source 830 so that the first electrode 802 and the fourth electrode 808 can perform current injection. That is, during the intrathoracic impedance measurement mode, a first switch 820 positioned between the first electrode 802 and the current source 830 (e.g., in a third wire 828) and a second switch 822 positioned between the fourth electrode 808 and the current source 830 (e.g., in a fourth wire 832) are closed. Further, a third switch 824 positioned between the first electrode 802 and the differential amplifier 814 (e.g., in a fifth wire 817) and a fourth switch 826 positioned between the fourth electrode and the differential amplifier 814 (e.g., in a sixth wire 819) are open.

In contrast, while operating in an ECG measurement mode shown in FIG. 8C, the first electrode 802 and the fourth electrode 808 are connected to the differential amplifier 814 and not to the current source 830. As shown, the first switch 820 positioned between the first electrode 802 and the current source 830 (e.g., in the third wire 828) and the second switch 822 positioned between the fourth electrode 808 and the current source 830 (e.g., in a fourth wire 832) are both open. The third switch 824 positioned between the first electrode 802 and the differential amplifier 814 (e.g., in the fifth wire 817) and the fourth switch 826 positioned between the fourth electrode and the differential amplifier 814 (e.g., in the sixth wire 819) are both closed during the ECG measurement mode. As such, the first electrode 802 and the second electrode 804 are electrically coupled at a junction between the first wire 816 and the fifth wire 817 to provide a single input to the differential amplifier 814 (the first voltage input V⁺), forming the first ECG electrode 810. Similarly, the third electrode 806 and the fourth electrode 808 are electrically coupled at a junction between the second wire 818 and the sixth wire 819 to provide a single input to the differential amplifier 814 (the second voltage input V⁻), forming the second ECG electrode 812. In this way, the first electrode 802 and the fourth electrode 808 can be used for either current injection (see FIG. 8B) or voltage sensing (see FIG. 8C), enabling the sensor circuit 850 to function as both an intrathoracic impedance sensor and an ECG sensor.

FIG. 9 shows a second exemplary electrode configuration 900 of a monitoring device. The monitoring device may be the monitoring device 100 of FIGS. 1A and 1B, for example. The second exemplary electrode configuration 900 includes a first electrode 902, a second electrode 904, a third electrode 906, and a fourth electrode 908 positioned in a housing 905. In the example shown in FIG. 9, each of the four electrodes is included in an intrathoracic impedance sensor, and two of the four electrodes are shared with an ECG sensor. In the present example, the first electrode 902 is a first impedance electrode (Imp1) used for current injection, the second electrode 904 is a second impedance electrode (Imp2) used for voltage measurement, the third electrode 906 is a third impedance electrode (Imp3) used for current injection, and the fourth electrode 908 is a fourth impedance electrode (Imp4) used for voltage measurement. For example, the first electrode 902 and the third electrode 906 are configured to inject a high frequency, low amplitude current into a subject, and the second electrode 904 and the fourth electrode 908 are configured to sense the resulting electrical potential from the current injected by the first electrode 902. Because the frequency used for impedance is higher than a bandwidth of ECG, the second electrode 904 and the fourth electrode 908 (e.g., used for measuring the electric potential) are also used to record ECG. Thus, the second electrode 904 is a first ECG electrode (ECG1) in addition to being the second impedance electrode, and the fourth electrode is a second EGC electrode (ECG2) in addition to being the fourth impedance electrode.

Turning now to FIG. 4, a schematic of a sensor unit 400 in the interior of a monitoring device is shown. The monitoring device may be the monitoring device 100 shown in FIGS. 1A and 1B, for example, and may include at least two sensors. A first sensor may comprise the electrical sensor 110 introduced in FIG. 1A, which is configured to measure electrical data from a subject via ECG electrodes. The sensor unit 400 may comprise an ECG transducer package 412 including the electrical sensor 110 and an analog-to-digital converter (ADC) 414 to digitize ECG signals detected by the ECG electrodes. The sensor unit 400 may comprise signal processing circuitry to filter and condition detected signals. The signal processing circuitry may comprise a filter 416. ECG data may be passed to an encoder 420. ECG signal processing circuitry may be implemented in the analog domain, in the digital domain, or both.

The ECG data may comprise single-lead ECG data. Single-lead ECG data may be obtained from one electrode that may be a ground and another electrode that may be a signal electrode. The voltage difference between the two leads may comprise analog ECG signal data. ECG data can be recorded as voltage as a function of time. As an alternative, the ECG data may comprise three-lead ECG data. The three-lead ECG data may be obtained from three electrodes, which may comprise, for example, right arm, left arm, and left leg electrodes.

The ECG data may comprise five lead ECG data. The five-lead ECG data may be obtained from five electrodes, which may comprise, for example, right arm, left arm, left leg, right leg, and central chest electrodes. The ECG data may comprise twelve-lead ECG data. The twelve-lead ECG data may comprise twelve electrodes, which may comprise, for example, right arm, left arm, left leg, right leg, central chest (sternum), sternal edge right fourth intercostal space, sternal edge left fourth intercostal space, between V2 and V4, mid-clavicular line left fifth intercostal space, between V4 and V6 left fifth intercostal space, and mid-axillary line left fifth intercostal space electrodes.

In some examples, the ECG data may comprise chest cavity, lung, and/or intra-thoracic impedance measurement data. The electrical data may comprise ECG data measured from a heart, lung, or other organ of a subject. The electrical data may comprise impedance data measured from a lung or intra-thorax of a subject (e.g., intrathoracic impedance data), such as described with respect to FIGS. 8 and 9. The electrical data may comprise ECG data measured from a bowel or other organ of a subject. The electrical sensor may comprise a voltage sensitivity of greater than or equal to about 1 microvolt, 10 microvolts, 0.1 millivolts (mV), 0.2 mV, 0.5 mV, 1 mV, 2 mV, 5 mV, 10 mV, 50 mV, 100 mV or more.

The second sensor modality may be an audio sensor, such as the audio sensor 112 described with respect to FIG. 1A. The audio sensor may comprise a piezoelectric sensor. The audio sensor may comprise an electric-based sensor. The audio sensor may be configured to collect audio data. The audio data may comprise audio data of a heart of a subject. The audio data may comprise audio data of a lung of a subject. The audio data may comprise audio data of a bowel or other organ of a subject. The audio sensor may comprise a part of an audio transducer package 402. The audio transducer package 402 may comprise an analog-to-digital converter 404 to digitize audio signals detected by the audio sensor. The sensor unit 400 may comprise signal processing circuitry to filter and condition detected signals, including the filter 406, to process the digitized audio signals. The audio data may be passed to the encoder 420. Audio signal processing circuitry may be implemented in the analog domain, in the digital domain, or both.

The audio sensor 112 may comprise a frequency response of about 20 Hertz (Hz) to 20 kilohertz (kHz). The audio sensor 112 may comprise a frequency response tuned to the low-frequency ranges between about 20 Hz and 2 kHz. The audio sensor 112 may comprise a response to frequencies greater than or equal to about 5 Hz, 10 Hz, 20 Hz, 50 Hz, 100 Hz, 200 Hz, 500 Hz, 1 kHz, 2 kHz, 5 kHz, 10 kHz, 20 kHz, 50 kHz, 100 kHz, or more. The audio sensor 112 may comprise a response to frequencies less than or equal to about 5 Hz, 10 Hz, 20 Hz, 50 Hz, 100 Hz, 200 Hz, 500 Hz, 1 kHz, 2 kHz, 5 kHz, 10 kHz, 20 kHz, 50 kHz, 100 kHz, or more. The audio sensor 112 may comprise a response tuned to a range between about 20 Hz and 2 kHz, between about 15 Hz and 10 kHz, between about 10 Hz and 10 kHz, between about 10 Hz and 20 kHz, etc.

The sensor unit 400 may further include a third sensor modality, such as the accelerometer 150 introduced in FIG. 1A. The accelerometer 150 may be three-axis accelerometer that measures proper acceleration, for example. The accelerometer 150 may be configured to detect both motion and a position of the monitoring device housing the sensor unit 400. The accelerometer 150 may use electrical, piezoelectric, piezoresistive, thermal (e.g., convective), or capacitive measurements, for example. In some examples, the accelerometer 150 is a microelectromechanical system (MEMS)-based accelerometer. The sensor unit 400 may comprise signal processing circuitry to filter and condition signals from the accelerometer 150, including the filter 408, to process acceleration signals measured by the accelerometer 150. The acceleration data may be passed to the encoder 420.

FIG. 5 shows a schematic of an interior of the monitoring device 100 including the sensor unit 400. Components that function the same as components described with respect to FIGS. 1A and 4 are numbered the same and will not be reintroduced. The monitoring device 100 may comprise electrical components configured to control the operation of the various sensors. For example, the monitoring device may comprise devices to store data (e.g., hard drive or memory), to transmit data, to convert analog data to digital data, to provide information on the functionality of the monitoring device, to control various aspects of data collection, etc. The monitoring device may comprise a microprocessor or microprocessing unit (MPU) 505. The microprocessor may be operably connected to a memory 510. The MPU can execute a sequence of machine-readable instructions, which can be embodied in a program or software. The instructions can be directed to the MPU, which can subsequently implement methods or components of methods of the present disclosure. Power may be supplied to the various components (the sensors, the microprocessors, the memory, etc.) by a battery 515. The battery 515 may be coupled to wireless charging circuitry.

The monitoring device may transmit data to a computing device over a network 530. The monitoring device may comprise a transceiver 520, such as a wireless transceiver, to transmit data to the computing device. The monitoring device may be connected to the Internet. The monitoring device may be connected to a cellular data network. The transceiver 520 may comprise a Bluetooth transceiver, a Wi-Fi radio, etc. Various wireless communication protocols may be utilized to convey data.

The monitoring device 100 may store data (e.g., ECG data, audio data, and/or data from any combination of the one or more sensors and/or any of the sensor modalities) locally on the monitoring device 100. In an example, the data may be stored locally on the memory 510 (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

The monitoring device may comprise electrical components necessary to process data from various sensors. For example, the monitoring device may comprise one or a plurality of the analog-to-digital converters (ADCs) 404 and 414 shown in FIG. 4. The one or a plurality of ADCs may sample the data from the various sensors such that electrical data is converted to a digital data stream. The monitoring device may comprise amplifier circuits and/or buffer circuits. The monitoring device may further comprise one or more components which compress the data of each sensor modality, such as the encoder 420 shown in FIG. 4. The monitoring device may further comprise one or more components which filter data of each sensor modality, including the filters 406, 408, and 416 shown in FIG. 4.

In some examples, the data (e.g., ECG data, audio data, intrathoracic impedance data, and/or acceleration data) may comprise a temporal resolution. The temporal resolution may be dictated by the sample rate of the one or more ADCs. For example, the time between samples of the one or more ADCs may be less than or equal to about 0.01 microsecond, 0.02 microsecond, 0.05 microsecond, 0.1 microsecond, 0.2 microsecond, 0.5 microsecond, 1 microsecond, 2 microseconds, 5 microseconds, 10 microseconds, 20 microseconds, 50 microseconds, 100 microseconds, 200 microseconds, 500 microseconds, 1 millisecond (ms), 2 ms, 5 ms, 10 ms, or more. Each of the ADCs may comprise its own sample rate which may be the same or different that other ADCs. Alternatively, one multi-channel ADC with a single sample rate may be used.

### Data Structures

FIG. 6 shows an example of a packet structure 600 for transmitting ECG and audio data. The monitoring device may transmit data via a wireless protocol, such as Bluetooth Low Energy protocol (BLE). The data may be transmitted in the packet structure 600. The transmitted data may comprise a reduced packet size in order to reduce power consumption. Packets may comprise multiple data streams such as sound (e.g., audio) data, ECG data, and command and control data associated with the operation of the monitoring device and its interaction with the computing device.

The data (e.g., ECG data, audio data and/or data from any combination of the one or more sensors and/or any of the sensor modalities) may be transmitted from the monitoring device to the computing device in a common packet. The common packet may convey multiple types of medical instrument and control data via a low-bandwidth and low-power BLE communication link that can be received by standard smartphones, tablets, or other computing devices described elsewhere herein. The packet structure may convey sound data 620, ECG data 625, and command and control data 610 simultaneously, with sufficient fidelity for clinical use, within a single BLE packet.

Each packet may comprise a byte length provided for by the BLE standard and packet intervals compatible with commodity BLE chipsets and computing devices. A data structure may provide an effective bitrate of more than or equal to about 1 kilobit per second (kbps), 5 kbps, 10 kbps, 20 kbps, 100 kbps, 1 gigabit per second (Gbps), 5 Gbps, 10 Gbps, 20 Gbps, 100 Gbps, 1 terabit per second (Tbps), or more.

The packet may include header bytes 605, command and control data 610, and data bytes. The data bytes may comprise sound data 620 and ECG data 625. The sound data bytes may be used for transmitting sound data from an audio sensor, such as the audio sensor described herein. The ECG data bytes may be used for transmitting electrical data from an electrical sensor, such as the ECG sensor described herein.

In an example, the audio sensor converts an audio signal, such as heart, lung, or bowel sound data, into an analog electrical signal. An analog-to-digital converter (ADC) samples the output of the audio sensor and generates a digital data stream. The ADC may sample at a rate of at least about 4 kHz with at least 16-bit samples which may yield a least a 64-kbps audio stream. Audio compression may be applied by adaptive differential pulse-code modulation (ADPCM) to yield a 4-bit audio stream at a 4-kHz rate. With an 8-millisecond (ms) packet interval, each packet includes audio having 32 4-bit audio samples. However, the packet interval may comprise a period of at least about 1 microsecond, 2 microseconds, 5 microseconds, 10 microseconds, 20 microseconds, 50 microseconds, 100 microseconds, 200 microseconds, 500 microseconds, 1 ms, 2 ms, 5 ms, 10 ms, 20 ms, 50 ms, 100 ms, or more.

In another example, the ADC may sample at a rate of at least about 500 kHz, 1 kHz, 2 kHz, 3 kHz, 4 kHz, 5 kHz, 6 kHz, 7 kHz, 8 kHz, 9 kHz, 10 kHz, 100 kHz, or more. The ADC may take at least 2-bit, 4-bit, 8-bit, 16-bit, 32-bit, 64-bit, 128-bit, 256-bit samples, or more. Audio compression may compress the audio stream by a factor of at least about 2, 3, 4, 5, 6, 7, 8, 9, 10, 15, 20, 25, 30, 35, 40, 45, 50, 60, 70, 80, 90, 100, 150, 200, 1000, or more.

Digital filters can be applied to the output of the ADC prior to the ADPCM encoder in order to reduce artifacts and distortion during the ADPCM compression process. In an example, filters may include low-pass filters to attenuate high-frequency components above the set frequency. The frequency of the low-pass filter may comprise at least about 20 Hz, 50 Hz, 100 Hz, 500 Hz, 1 kHz, 2 kHz, 3 kHz, 4 kHz, 5 kHz, 6 kHz, 7 kHz, 8 kHz, 9 kHz, 10 kHz, 15 kHz, 20 kHz, or more. In an example, filters may include high-pass filters to attenuate low-frequency components below the set frequency. The frequency of the high-pass filter may comprise at least about 20 Hz, 50 Hz, 100 Hz, 500 Hz, 1 kHz, 2 kHz, 3 kHz, 4 kHz, 5 kHz, 6 kHz, 7 kHz, 8 kHz, 9 kHz, 10 kHz, 15 kHz, 20 kHz, or more. In other examples, the filters may comprise band pass filters, Fourier filters, or other filters. Frequency range limitations may be beneficial for purposes of medical diagnostics to reduced compression noise and artifacts from the ADPCM encoder.

ECG signals may be sampled by an analog-to-digital converter (ADC). The ECG signals may be sampled by the same ADC as the audio signals or a separate ADC. The audio ADC and the ECG ADC may comprise substantially similar characteristics. Alternatively, the sampling characteristics of the ECG ADC may be adapted for electrical data. In an example, the ADC may sample at a rate of at least about 500 Hz, 1 kHz, 2 kHz, 3 kHz, 4 kHz, 5 kHz, 6 kHz, 7 kHz, 8 kHz, 9 kHz, 10 kHz, 100 kHz, or more. The ADC may take at least about 2-bit, 4-bit, 8-bit, 16-bit, 32-bit, 64-bit, 128-bit, 256-bit samples or more. However, the packet interval may comprise a period of at least about 1 microsecond, 2 microseconds, 5 microseconds, 10 microseconds, 20 microseconds, 50 microseconds, 100 microseconds, 200 microseconds, 500 microseconds, 1 millisecond (ms), 2 ms, 5 ms, 10 ms, 20 ms, 50 ms, 100 ms, 500 ms, 1 second, or more.

The ECG data may be compressed. For example, compression may be applied by the ADPCM or another data compression method. Data compression may compress the ECG data stream by a factor of at least about 2, 3, 4, 5, 6, 7, 8, 9, 10, 15, 20, 25, 30, 35, 40, 45, 50, 60, 70, 80, 90, 100, 150, 200, 1000, or more. The ECG data may be filtered. In an example, filters may include low-pass filters to attenuate high-frequency components above the set frequency. The frequency of the low-pass filter may comprise at least about 20 Hz, 50 Hz, 100 Hz, 500 Hz, 1 kHz, 2 kHz, 3 kHz, 4 kHz, 5 kHz, 6 kHz, 7 kHz, 8 kHz, 9 kHz, 10 kHz, 15 kHz, 20 kHz, or more. In an example, filters may include high-pass filters to attenuate low-frequency components below the set frequency. The frequency of the high-pass filter may comprise at least about 20 Hz, 50 Hz, 100 Hz, 500 Hz, 1 kHz, 2 kHz, 3 kHz, 4 kHz, 5 kHz, 6 kHz, 7 kHz, 8 kHz, 9 kHz, 10 kHz, 15 kHz, 20 kHz, or more. In other examples, the filters may comprise band pass filters, Fourier filters, or other filters.

The command-control data 610 (alternatively, the command and control data 610) may comprise command data and/or control data. The command-control data 610 may be implemented in header bits. In an example, a header bit may comprise different command-control data for different packets with the same or similar bit size. A header bit or bits may be utilized to indicate which of multiple types of command-control data are conveyed within associated packet bit positions. For example, a header bit may include volume level, battery level, link integrity data, a time stamp, sequence number, etc.

Depending on the application, at least some or all of the command-control data 610 may be sent in the header of every packet. For example, volume information may be sent at a fraction of the sample rate of the sensor data. A piece of command-control data stored in one or more header bits may be sent at rate less than the sensor data of at least about a factor of 2, 3, 4, 5, 6, 7, 8, 9, 10, 15, 20, 25, 30, 35, 40, 45, 50, 60, 70, 80, 90, 100, 150, 200, 1000, or more.

In examples where a given piece of command-control data is sent at a lower rate than the sample data, a single header bit may be used to carry more than one type of data. For example, a piece of header data of type A may be sent in every-other packet in header bit 1, and a second piece of header data of type B may be sent in header bit 1 in the rest of the packets. By this method, the number of header bits used may be significantly reduced. Multiple header bits can be utilized to enable greater numbers of command-control data content types to be conveyed within a given packet bit position.

A computing device may display a warning on a user interface of the computing device. The warning may be indicative of a compromise in a link between the monitoring device and the computing device. It may be beneficial in medical applications to verify a link integrity. It may be desirable for devices to rapidly and reliable alert the user when a data transmission quality problem arises. A user may then remedy equipment problems and ensure that anomalous results are attributed to instrumentation error rather than the patient being monitored.

A rolling packet sequence may be inserted into the common packet structure. A rolling packet structure may comprise a link verification mechanism. The rolling packet structure may be disposed in the header bit of the packet structure. Predetermined bits within the header may be allocated to a rolling packet sequence indicator. The processor of the monitoring device may construct consecutive packets to increment through a rolling multi-bit packet sequence value. The computing device can decode the packet sequence value to verify that consecutive packets are received with sequentially incrementing packet sequence values.

A computing device may receive a sequential data packet having a non-sequential rolling packet sequence. In this example, the monitoring device may determine that a link has been compromised. The monitoring device may alert a user, such as a subject or a medical professional using an indication on the monitoring device. An indication on the monitoring device may comprise a light, a sound, a vibration, or other approach to alert a subject or another user. Additionally or alternatively, the monitoring device may indicate a compromised link through a communication with a remote computing device. In some examples, the monitoring device may send an alert to a remote computing device to alert a remote user.

Data may be presented on a user interface of a computing device in substantially real-time. The packet structure may comprise additional header bits 615 in order to periodically send control data to a computing device to assure quality of data stream and synchronization between data streams. The runtime header bits may comprise a sequence number and/or a timestamp. The runtime header bits may include a reset bit to initialize or re-initialize the data compression. The runtime header bit may include device status information including battery charge, filtering state, volume level, and/or temperature. In some examples, the runtime header bits may comprise a portion of the command control data. The runtime header bits may comprise run time protocol. The runtime header bits may vary from packet to packet. The runtime header bits may vary based on a time of measurement. For example, the runtime header bit may change to provide an update of the status of a measurement, the monitoring device, a battery level, etc. For example, the runtime header data may be sent at a lower rate than the sample data to reduce size of the data stream.

### Trained Algorithms

Methods and systems of the present disclosure can be implemented by way of one or more algorithms. An algorithm can be implemented by way of software upon execution by a processing unit of the monitoring device, the computing device, or a connected server. The algorithm may analyze data (e.g., ECG data, audio data, intrathoracic impedance data, accelerometer data, and/or data from any combination of the one or more sensors and/or any of the sensor modalities) in order to provide an output indicative of a state or condition of an organ or organ system, such as a heart, a lung, or a bowel of a subject. The algorithm can, for example, be used to process a suitable combination of data measured using the monitoring device (e.g., ECG data, audio data, intrathoracic impedance data, accelerometer data, and/or data from any combination of the one or more sensors and/or any of the sensor modalities) to determine the physiological or biological state or condition of an organ or organ system the subject, such as heart, lung, bowel, or any other organ or organ system.

In an example, the data may be processed by one or more computer processors of the computing device, described elsewhere herein. The data may comprise ECG data and audio data. The data may further comprise intrathoracic impedance data and accelerometer data. In some examples, the data may further comprise data collected using one or more sensors, such as any data collected using any sensor listed anywhere herein. An algorithm can be implemented upon execution by the processor of the monitoring device. Additionally or alternatively, an algorithm can be implemented upon execution by the processor of a connected server. In some examples, the trained algorithm may process the data on the computing device, the monitoring device, and/or both. The algorithm may process the data in a cloud system, such as a distributed cloud computer system. The algorithm may process the data on a computing device, such as a smartphone, PC, tablet, or any other kind of device.

The algorithm may be a trained algorithm. The algorithm may be trained by a supervised or a guided learning method. For example, the trained algorithm may comprise a support vector machine, a decision tree, a stochastic gradient descent method, a linear discriminate analysis method, etc. Alternatively, the algorithm may be trained by an unsupervised learning method. For example, the algorithm may comprise clustering method, a decomposition method, etc. In an example, the learning method may be semi-supervised. Examples of learning algorithms include support vector machines (SVM), linear regression, logistic regression, naive Bayes, linear discriminant analysis, decision trees, k-nearest neighbor algorithm, random forests, and neural networks (or multilayer perceptron).

The algorithm may be trained by a training set that is specific to a given application, such as, for example classifying a state or condition (e.g., a disease). The algorithm may be different for heart disease and lung disease, for example. The algorithm may be trained for application in a first use case (e.g., arrhythmia) using a training set that is different than training the algorithm for application in a second use case (e.g., pneumonia). The algorithm may be trained using a training set of subjects with known states or conditions (e.g., disorders). In some examples, the training set (e.g., type of data and size of the training set) may be selected such that, in validation, the algorithm yields an output having a predetermined accuracy, sensitivity and/or specificity (e.g., an accuracy of at least 90% when tested on a validation or test sample independent of the training set).

The trained algorithm may be a neural network. The neural network may comprise an unsupervised learning model or a supervised learning model. The audio and/or ECG data may be input into the neural network. Additional information such as age, gender, recording position, weight, or organ type may be inputted into the neural network. The neural network may output the likelihood of a pathology or disease, a disease severity score, an indication of lung dysfunction, an indication of heart failure, an indication of atrial fibrillation, an indication of different types of heart murmur, such as mitral regurgitation, tricuspid regurgitation, or other diseases or healthy states. In some examples, the neural network may be used to analyze the audio data, the ECG data, or both the audio and the ECG data.

The neural network may further use ECG data to cancel noise in audio data and create a clean representation of the audio data or otherwise process the audio data. In some examples, the neural network may use the accelerometer data to gate inputs into the processing of the ECG data and the audio data. For example, the neural network may not process ECG data and audio data recorded while the accelerometer data detects motion. The neural network may create different combinations of the audio and ECG data. For example, the audio signals recorded for a heartbeat of a subject can be noisy due to subject motion, device motion, or another reason. On a spectrogram of a waveform of the heartbeat can include peaks that are attributed to ambient noises even after sound filtering. The ECG data can be used to further remove noises after sound filtering in the audio data and provide a clean representation of the heart beat recorded. This may be performed by using the ECG data to trigger averaging of the audio data by comparing the QRS complex recorded by the ECG to the recorded audio signals.

Referring briefly to FIGS. 11A and 11B, the QRS complex represents a combination of three of the graphical deflections seen on a typical ECG and can characterize a shape of a heartbeat signal. For example, an R-peak from the QRS complex (FIG. 11A) represents an ending of atrial contraction and a beginning of ventricular contraction. An audio waveform of a heartbeat typically represents a “lub-dub” sound. The “lub” sound occurs during the early phase of ventricular contraction and is produced by closing of the atrioventricular valves. Further, the “dub” sound occurs when the ventricles relax. Referring to FIG. 11B, time 0 of an audio waveform of a heartbeat can be matched to an R-peak in a QRS complex, thus peaks in the spectrogram of the waveform caused by noises can be disregarded.

A method to filter ECG signal and/or data is disclosed herein. In some examples, the trained algorithm may be configured to filter ECG signal and/or data. The ECG data may be filtered with low latency by identifying/isolating R-peaks. In some examples, R-peaks may be identified in real-time or substantially real-time and be used to filter ECG data or signals. In some examples, multiple filters, such as at least 0, 1, 2, 3, 4, 5, 6, 7, 8, 9, 10, or more filters may be applied to ECG signals. In an example, a filter may be applied to R-peaks, while another filter is applied to the rest of the ECG signal. The filter applied to R-peaks may be different from the filter applied to the rest of the ECG signal (other than R-peaks). Alternatively, the filter applied to R-peaks may be the same as the filter applied to the rest of the ECG signal/data.

The methods may comprise processing data (e.g., audio data and/or ECG data, or other types of data) it a suitable way. In some examples, the trained algorithm may be configured to process data. Such method may involve using R-peaks. In some examples, the method may further comprise providing, showing, or presenting the processed data and/or the non-processed data to the user and/or the care provider. For example, an average envelope of the audio data may be computed. Such average envelope of the audio data may be triggered by R-peaks, and may be further used to compute, simulate, calculate or find an average heart beat/sound of the subject. The average heart sound of the subject may be further shown, presented, displayed, or otherwise conveyed to the user or healthcare provider in any form, such as in form of digital and/or electronic data, audio or sound (e.g., through the monitoring device, computing device, earpieces, other device parts, and/or any combinations thereof), graph, display, or any other suitable format. In some examples, the audio of the heart beat may be further added to the average envelope (of the audio data), for example, if/when such data does not show presence of artifacts. Alternatively, the audio of the heart beat may not be added to the average envelope (of the audio data). Similarly, an average of the ECG data (e.g., average ECG waveform) may be computed. The average ECG waveform may be triggered by R-peaks. The average ECG waveform may be further presented or provided to the user, such as by displaying the shape of the computed ECG waveform to the user. In some examples, the average ECG waveform may further be added to an average envelope of the ECG data, for example, if the average ECG waveform does not indicate a presence of artifacts and/or abnormal beats (e.g., heart beat).

Further, the neural network may be used to screen for a certain state or condition of a subject. The neural network may calculate a combined score to provide a quantitative metric for a state or condition of a subject comprising the combination of several metrics such as recorded ECG data, recorded audio data, data from other sensors such as a weight scale or an implantable sensor, user-input data, or data from other sources. Implantable sensors comprise implantable devices capable of providing real-time hemodynamic data such as Heart Failure (HF) systems further comprising CardioMEMS, right ventricular (RV) sensors, pulmonary artery (PA) sensors, and left atrial pressure (LAP) sensors, diagnostic features in implantable cardiac resynchronization therapy (CRT) devices and implantable cardioverter defibrillator (ICD) devices. Combined scores may directly or indirectly predict a state or condition of the subject such as detecting a low ejection fraction or normal ejection fraction of the subject. Ejection fraction (EF) is a measurement, expressed as a percentage, of how much blood the left ventricle pumps out with each contraction. In a healthy state, an ejection fraction of a subject may be in the range between 55% and 70%. Low ejection fraction, or low EF, is the term used to describe an ejection fraction of a subject if it falls below 55%. Data and analysis from a neural network single lead ECG waveform, the presence and intensity of the third heart sound (S3) as detected by audio alone, ECG data alone, or a combination thereof, and the value of electromechanical activation time (EMAT) and can all be correlated to determine ejection fraction. The neural network may combine all of the three metrics to arrive at a combined score which is proportional to or related to the ejection fraction of the subject. In another example, the combined score can predict pulmonary artery pressure as measured by an implantable sensor like the CardioMEMS HF system.

In some examples, audio recordings may be manually labeled or annotated by physicians. The audio recordings may be manually labeled or annotated by data-scientists. In some examples, ECG data may be manually labeled or annotated by physicians. The ECG data may be manually labeled or annotated by data-scientists. The labeled data may be grouped into independent training, validation, and/or test data sets. The labeled data may be grouped such that all recordings from a given patient are included in the same set. The neural network may comprise a training dataset which has been classified. The neural network may be trained on a set of data comprising audio and ECG data with an assigned classification. A classification may comprise a dysfunction score. A classification may comprise a known diagnosis or determination. Alternatively, a classification may be assigned by a decomposition method such as singular value decomposition, principle component analysis, etc.

The trained algorithm may be configured to accept a plurality of input variables and to produce one or more output values based on the plurality of input variables. The plurality of input variables may comprise ECG data and/or audio data. The plurality of input variables may also include clinical health data of a subject. The one or more output values may comprise a state or condition of a subject (e.g., a state or condition of a heart, lung, bowel, or other organ or organ system of the subject). Further, in some examples, the trained algorithm may give more weight to certain characteristics of a state or condition. For example, for detecting heart murmur, the trained algorithm may be able to analyze identified sounds including S1, S2, and suspected murmurs. The trained algorithm may be able to analyze ECG data along with parameters such as, EMAT, left ventricular systolic twist (LVST), S3 strength, S4 strength, and SDI. For calculating hear rate and heart rate variability and the detection of atrial fibrillation, the trained algorithm may be able to analyze ambulatory ECG data and single-lead ECG signals.

The trained algorithm may comprise a classifier, such that each of the one or more output values comprises one of a fixed number of possible values (e.g., a linear classifier, a logistic regression classifier, etc.) indicating a classification of a state or condition of the subject by the classifier. The trained algorithm may comprise a binary classifier, such that each of the one or more output values comprises one of two values (e.g., {0, 1}, {positive, negative}, or {high-risk, low-risk}) indicating a classification of the state or condition of the subject. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the state or condition of the subject.

The output values may comprise descriptive labels, numerical values, or a combination thereof. Some of the output values may comprise descriptive labels. Such descriptive labels may provide an identification or indication of a state or condition of the subject, and may comprise, for example, positive, negative, high-risk, intermediate-risk, low-risk, or indeterminate. Such descriptive labels may provide an identification of a treatment for the state or condition of the subject, and may comprise, for example, a therapeutic intervention, a duration of the therapeutic intervention, and/or a dosage of the therapeutic intervention suitable to treat the state or condition of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. As another example, such descriptive labels may provide a prognosis of the state or condition of the subject. As another example, such descriptive labels may provide a relative assessment of the state or condition of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the state or condition of the subject. Some numerical values may be mapped to descriptive labels, for example, by mapping 1 to “positive” and 0 to “negative.”

Some of the output values may be assigned based on one or more cutoff values. For example, a binary classification of subjects may assign an output value of “positive” or 1 if the subject has at least a 50% probability of having the state or condition. For example, a binary classification of subjects may assign an output value of “negative” or 0 if the subject has less than a 50% probability of having the state or condition. In this example, a single cutoff value of 50% is used to classify subjects into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of subjects may assign an output value of “positive” or 1 if the subject has a probability of having the state or condition of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of subjects may assign an output value of “positive” or 1 if the subject has a probability of having the state or condition of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of subjects may assign an output value of “negative” or 0 if the subject has a probability of having the state or condition of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of subjects may assign an output value of “negative” or 0 if the subject has a probability of the state or condition of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of subjects may assign an output value of “indeterminate” or 2 if the subject is not classified as “positive”, “negative”, 1, or 0. In this example, a set of two cutoff values is used to classify subjects into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify subjects into one of n+1 possible output value, where n is any positive integer.

The trained algorithm may be trained with a plurality of independent training samples. Each of the independent training samples may comprise a dataset of ECG data and/or audio data collected from a subject at a given time point, and one or more known output values corresponding to the subject. Independent training samples may comprise datasets of ECG data and/or audio data and associated output values obtained or derived from a plurality of different subjects. Independent training samples may comprise datasets of ECG data and/or audio data and associated output values obtained at a plurality of different time points from the same subject (e.g., on a regular basis such as weekly, biweekly, or monthly). Independent training samples may be associated with presence of the state or condition (e.g., training samples comprising datasets of ECG data and/or audio data and associated output values obtained or derived from a plurality of subjects known to have the state or condition). Independent training samples may be associated with absence of the state or condition (e.g., training samples comprising datasets of ECG data and/or audio data and associated output values obtained or derived from a plurality of subjects who are known to not have a previous diagnosis of the state or condition or who have received a negative test result for the state or condition). A plurality of different trained algorithms may be trained, such that each of the plurality of trained algorithms is trained using a different set of independent training samples (e.g., sets of independent training samples corresponding to presence or absence of different states or conditions).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise datasets of ECG data, intrathoracic impedance data, and/or audio data associated with presence of the state or condition and/or datasets of ECG data, intrathoracic impedance data, and/or audio data associated with absence of the state or condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the state or condition. In some embodiments, the dataset of ECG data, intrathoracic impedance data, and/or audio data is independent of samples used to train the trained algorithm.

The trained algorithm may be trained with a first number of independent training samples associated with presence of the state or condition and a second number of independent training samples associated with absence of the state or condition. The first number of independent training samples associated with presence of the state or condition may be no more than the second number of independent training samples associated with absence of the state or condition. The first number of independent training samples associated with presence of the state or condition may be equal to the second number of independent training samples associated with absence of the state or condition. The first number of independent training samples associated with presence of the state or condition may be greater than the second number of independent training samples associated with absence of the state or condition.

The data using may be modeled using a deep convolutional neural network architecture. The convolutional neural network may classify audio segments, intrathoracic impedance data segments, and/or ECG data segments over a measurement time. For example, the audio segments may be about 5 seconds long. For example, the audio segments may be within a range between about 0.1 second and 1 minute. The audio segments may be within a range between 1 second and 10 minutes. The audio seconds may be less than or equal to about 6 months, 3 months, 2 months, 1 months, 3 weeks, 2 weeks 1 week 6 days, 5 days, 4 days, 3 days, 1 day, 12 hours, 6 hours, 5 hours, 4 hours, 3 hours, 2 hours, 1 hour, 30 minutes, 10 minutes, 5 minutes, 1 minute, 30 seconds, 10 seconds, 5 seconds, 1 second, 100 milliseconds, or less. As an alternative or in addition, the audio segments may be at least about 100 milliseconds, 1 second, 5 seconds, 10 seconds, 20 seconds, 30 seconds, 1 minute, 5 minutes, 10 minutes, or more. Alternatively, such time period may be at most about 6 months, 3 months, 2 months, 1 months, 3 weeks, 2 weeks 1 week 6 days, 5 days, 4 days, 3 days, 1 day, 12 hours, 6 hours, 5 hours, 4 hours, 3 hours, 2 hours, 1 hour, 30 minutes, 10 minutes, 5 minutes, 1 minute, 30 seconds, 20 seconds, 10 seconds, 5 seconds, or less. The time period may be from about 1 second to 5 minutes, or from about 5 seconds to 2 minutes, or from about 10 seconds to 1 minute.

The model may comprise a number of layers. The number of layers may be between about 5 and 1000. The model may comprise at least about 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 1000, 10000, 100000, 10000000, or more layers.

Each layer may comprise a one-dimensional convolution. Each layer may comprise a multidimensional convolution. Each layer may comprise a convolution with a dimensionality of at least about 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, or more. Each layer may comprise a stride and/or a padding. The stride and/or the padding may be adjusted such that the size of the output volume is manageable. In some examples, the padding may be zero. In some examples, the padding may be non-zero.

Each layer may comprise a rectified linear unit (ReLU) layer or an activation layer. However, in some examples, a hyperbolic tangent, sigmoid, or similar function may be used as an activation function. Each layer may be batch normalized. In some examples, the layers may not be batch normalized. In some examples, the network may comprise a pooling layer or a down sampling layer. In some examples, the pooling layer may comprise max pooling, average pooling, and L2-norm pooling, or similar. In some examples, the network may comprise a dropout layer. In some examples, the neural network comprises a residual neural network or ResNet. In some examples, the neural network may comprise skip-layer connections, or the neural network may comprise residual connections. Without being limited by theory, residual neural networks may help alleviate the vanishing gradient problem.

The neural network may be implemented using a deep learning framework in Python. In some examples, the neural network may use Pytorch & Torch, TensorFlow, Caffe, RIP, Chainer, CNTK, DSSTNE, DYNEt, Gensim, Gluon, Keras, Mxnet, Paddle, BigDL or similar deep learning framework. The neural network may be trained using TensorFlow, Google Cloud Machine Learning, Azure Machine Learning, Theano, GCT, Chainer, or similar.

The model may trained for a number of epochs, which may be at least about 1, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 600, 700, 800, 900, 1000, 10000, 100000, or more. In some examples, regularization hyperparameters may be varied and evaluated based number of correct predictions on the validation set to determine a model with satisfactory performance. Model parameters may be iterated to achieve effective performance using an algorithm. The model parameters may use a stochastic gradient decent algorithm. The model parameters may be varied using an adaptive gradient algorithm, adaptive moment estimation, Adam, root mean square propagation, or similar. After each epoch, the classifier loss may be evaluated based on a validation set. The step size may be annealed by a factor of at least 2 after the validation loss has plateaued. In some examples, the step size may not be annealed. The model parameters from the epoch with the lowest overall validation loss may be selected for the model.

The model may be subsequently used to evaluate audio data alone, ECG data alone, intrathoracic impedance data, or a combination of two or more data types to determine the presence or absence of a state or condition of an organ, such as a murmur of a heart. For example, the model may be used to detect a murmur of a heart based on the above criteria. The model may be further used to determine the type of the murmur detected. Heart murmurs may comprise systolic murmurs, diastolic murmurs, continuous murmurs, holosystolic or pansystolic murmurs, and plateau or flat murmurs. In some examples, the audio data may split into segments, as described herein with respect to training. The audio data over a period may be analyzed independently by the network. The network may output a probability of state or condition of a heart for each segment. These probabilities may then be averaged across all or a fraction of the segments. The average may then be thresholded to make a determination of whether a state or condition of an organ, such as a heart murmur is present.

Features of the audio data, the ECG data, or a combination of the audio and ECG data can be used to classify or determine a state or condition of the heart of a subject. Features of the recorded audio may comprise the intensity of audio frequency data, the pitch of the audio frequency data, change in the intensity of the audio frequency data over time also known as the shape of the audio frequency data, the location the signals are most intensely detected, the time during the audio cycle of the heart where the signals are detected, tonal qualities, and more. Further, features of the ECG diagram may comprise average numbers or standard deviation numbers of PR segments, ST segments, PR intervals, QRS intervals, ST intervals, or QT intervals.

For example, the state or condition of the heart of the subject may be correlated with a magnitude and a duration of the audio data within a frequency band of the audio data. A state or condition of a heart may be based on the magnitude and duration of audio in a specific frequency range. Particularly, the severity of a murmur of a heart may be correlated with the magnitude and duration of audio in a specific frequency band that is correlated with specific disease states. The magnitude or intensity of the audio may comprise a 6-point scale in evaluating heart murmurs. For example, absence of a heart murmur is graded as 0/6. Murmurs that are clearly softer than the heart sounds are graded 1/6. Murmurs that are approximately equal in intensity to the heart sounds are graded 2/6. Further, murmurs that are clearly louder than the heart sounds are graded 3/6. For score 4/6, the murmurs are easily audible and associated with a thrill. Moreover, a grade 6/6 is extremely loud and can be heard with a stethoscope even when slightly removed from the chest. Many other characteristics of sound can be used to evaluate heart murmurs as well. The pitch of the audio can be used to evaluate heart murmurs by classifying pitches as high, medium, or low. Tonal qualities such as blowing, harsh, rumbling, booming, sharp, dull or musical can also be used to evaluate heart murmurs.

The state or condition of the heart of the subject may be correlated with a certain audio frequency at a certain time during the audio cycle of the heart. The audio cycle of heart comprises normal heart sounds S1 and S2. The duration of time between S1 and S2 is called systole. The duration of time between S2 and the next S1 in the cycle is called diastole. Extra heart sounds S3 and S4 may be detected during the audio cycle of the heart which may be correlated with a state or condition of the heart, such as a diagnosis. Heart sounds or signals detected during the systole may be correlated with systolic conditions. Heart sounds or signals detected during the diastole may be correlated with diastolic conditions. Heart sounds may comprise continuous sounds during the audio cycle of the heart which may be correlated with certain states or conditions of the subject. The state or condition of the heart of the subject may be correlated with the change in the intensity of the audio signals over time. The intensity of audio signals over time can also be demonstrated by various shapes. Shapes of audio signals, which can also be used to classify murmurs, comprise crescendo, decrescendo, crescendo-decrescendo, or plateau, also known as flat. Crescendo signals increase over time. Decrescendo signals decrease over time. Crescendo-decrescendo means the intensity of the signals initially increases over time, but after a certain time starts to decrease over time. Plateau or flat signals remain stable over time.

The state or condition of the heart of the subject may comprise a measurement of the electro-mechanical activation time, which may correspond to or correlate with the time difference between “Q” wave of the ECG and the first heart sound “S 1” and an output indicative of such state or condition may be provided accordingly. The output indicative of the state or condition of the subject (e.g., heart of the subject) may comprise a measurement of the pre-ejection period, which may correspond to or correlate with the time difference between the “Q” wave of the ECG and opening of the aortic valve. The output indicative of the state or condition of the subject (e.g., heart of the subject) may comprise determining a presence or absence of bradycardia or tachycardia. Such condition may be detected through measuring and/or analyzing/processing the heart audio data, (phonocardiogram/PCG), ECG data, and/or both. The output indicative of the state or condition of the subject (e.g., the heart of the subject) may comprise determining a presence or absence of pulmonary hypertension or pulmonary arterial hypertension.

FIG. 12 shows examples of various heart murmurs. Panel 1210 depicts a presystolic crescendo murmur of mitral or tricuspid stenosis. Panel 1220 depicts a holosystolic (pansystolic) flat/plateau murmur of mitral or tricuspid regurgitation or of ventricular septal defect. Panel 1230 depicts a crescendo-decrescendo aortic ejection murmur beginning with an ejection click and fading before the second heart sound. Panel 1240 depicts a crescendo-decrescendo systolic murmur in pulmonic stenosis spilling through the aortic second sound, pulmonic valve closure being delayed. Panel 1250 depicts a decrescendo aortic or pulmonary diastolic murmur. Panel 1260 depicts a long diastolic murmur of mitral stenosis after an opening snap. Panel 1270 depicts a short mid-diastolic inflow murmur after a third heart sound. Panel 1280 depicts a continuous murmur of patent ductus arteriosus.

The state or condition of the heart may be correlated with the location in a subject where the signal is most loudly or intensely detected. For example, the audio may be most intensely detected in the aortic region, the pulmonic region, the mitral region also known as the apex, the tricuspid region, the left sternal border in the intercostal space of the subject or along the left side of the sternum or other locations in the subject. Therefore, these locations may be the best for detecting a heart murmur.

In addition, features of audio data, ECG data, intrathoracic impedance data, or a combination of two or more of audio and ECG data can also be used to classify and evaluate other states or conditions of a subject. Examples of states or conditions of a subject comprise aortic stenosis, pulmonic stenosis, mitral regurgitation, tricuspid regurgitation, mitral valve prolapse, aortic regurgitation, pulmonic regurgitation, mitral stenosis, tricuspid stenosis, volume overload, pressure overload or atrial gallop. Aortic stenosis is a systolic heart murmur correlated with a crescendo-decrescendo audio frequency detected after the first normal heart sound S1 and before the second normal heart sound S2 in the audio cycle of the heart, most intensely detected in the aortic region in the intercostal space of the subject. Pulmonic stenosis is a systolic heart murmur correlated with a crescendo-decrescendo audio frequency detected after the first normal heart sound S1 and before the second normal heart sound S2 in the audio cycle of the heart most intensely detected in the aortic region in the intercostal space of the subject. Mitral regurgitation is a holosystolic heart murmur correlated with a plateau/flat audio frequency detected after the first normal sound S1, late in the systole, most intensely detected in the mitral region/apex of the intercostal space of the subject. Tricuspid regurgitation is a holosystolic murmur correlated with a plateau/flat audio frequency detected after the first normal sound S1 and before the second normal sound S2 in the audio cycle of the heart, most intensely detected in the tricuspid region of the intercostal space of the subject. Mitral valve prolapse is a systolic murmur associated with a mid-systolic non-ejection click most intensely detected in the mitral region/apex of the intercostal space of the subject. Aortic regurgitation is a diastolic heart murmur correlated with a decrescendo audio frequency detected after the second normal heart sound S2 most intensely detected in the left sternal border in the intercostal space of the subject. Pulmonic regurgitation is a diastolic heart murmur correlated with a decrescendo audio frequency after the second normal heart sound S2 most intensely detected along the left side of the sternum. Mitral stenosis is a diastolic heart murmur correlated with an audio frequency after the second normal heart sound most intensely detected in the mitral area or the apex, also correlated with an opening snap followed by a mid-diastolic rumble or rumbling sound in the diastole during the audio cycle of the heart. Tricuspid stenosis is a diastolic heart murmur correlated with an audio frequency after the second normal heart sound S2 most intensely detected in the tricuspid area in the intercostal space of the subject.

The frequency may correlate with turbulent blood flow caused by a narrowing of a valve in the heart. The frequency may correlate with blood flow caused by a hole between two chambers of the heart. The frequency may correlate with blood flow through a narrowed coronary artery. The frequency may correlate with regurgitation in the blood flow. The frequency may correlate with impaired cardiac muscle function. The frequency may correlate with the ECG data to indicate cardiac muscle function. The frequency data may comprise a correlation with heart failure including congestive heart failure diagnosis or other cardiovascular conditions.

The ECG, intrathoracic impedance, and/or audio data may comprise features associated with known pathologies. Features associated with known pathologies may comprise diagnostic features. The ECG, intrathoracic impedance, and/or audio data may be reduced in size by determining a set of diagnostic features from the data. The diagnostic features may comprise factors known to effect diagnostic outcomes such as an average or a standard deviation of time interval between heart beats, the average or standard deviation in an amplitude of an ECG signal associated with a heart contraction, etc. An average or standard deviation of one or more of a QT interval, ST segment, PR interval, PR segment, QRS complex, a width of the QRS interval, the QTC, interval, etc. Alternatively, a set of features may be determined by a spectral decomposition of an ECG and/or audio data set. In an example, a diagnostic feature is assigned by a user, such as a health care provider. The ECG data may be correlated with atrial fibrillation through the presence or absence of characteristic ECG waves. The ECG data may be correlated with heart failure through a relationship with the heart sounds. The ECG data may be correlated with systolic function in the heart through wave lengths or ECG interval durations. The ECG data may be correlated with fluid status in the lungs through intra-thoracic impedance measurements.

An output indicative of the physiological or biological state or condition of the heart of the subject may then be provided on the computing device. The output may be an alert indicative of an adverse state or condition of the heart. In an example, an output indicative of the state or condition of the heart of the subject may comprise a presence or absence of a low ejection fraction of a left ventricle of the heart of the subject or a normal ejection fraction in the heart of the subject. An output of the state or condition of a heart of a subject may comprise an indicator of systolic function. The output indicative of the state or condition of the subject may comprise information about the width of the QRS interval, the ST interval, the PR interval, the QT interval, the QTc interval, and more. The output may comprise information indicative of a presence or absence of prolonged (or long) QT syndrome. The output may comprise determining the RR interval and/or heart rate of the heartbeat. Such indication may be based on data collected from the monitoring device, such as from the one or more sensors of the monitoring device, such as ECG sensor, audio sensor, other sensors listed anywhere herein, and/or any combination thereof.

The output indicative of the state or condition of the subject may comprise determining a presence or absence of atrial fibrillation. The output can consist of atrial fibrillation, information about sinus rhythm such as normal or abnormal sinus rhythm, trigeminy, bigeminy, premature ventricular contraction, premature atrial contraction, other conditions, and/or any combination thereof. The output indicative of the state or condition of the subject may comprises determining a presence or absence of hypertrophic cardiomyopathy.

In some examples, an output indicative of a state or condition of the subject may comprise “poor signal” or “poor signal quality.” For example, in some examples, data collected from a sensor of the one or more sensors of the monitoring device may have recorded a weak signal, and output may be indicative of such weak signal. The user may identify that they need to repeat the measurement or data recording or capture using the monitoring device for a better signal, so that another output indicative of the state or condition of the subject could be provided. In some examples, the output may comprise “unclassified.” For example, the data collected using the monitoring device may not be a good match for a known condition or such condition may not be present in the database or for any reason, the state or condition of the subject may not be classified. As such, an output may indicate that the state or condition of the subject is unclassified.

Determining the state or condition of the subject may comprise determining the state or condition of an organ of the subject, such as a heart or a lung of the subject. The state or condition of various parts of a body of the subject may be determined. Determining the state or condition of a heart of a subject may comprise a diagnosis or determination of low ejection fraction, normal ejection fraction, congestive heart failure, heart failure risk score, heart murmur, arrhythmia, heart blockage, ischemia, infarction, pericarditis, hypertrophy, or determining or predicting the pressure of the pulmonary artery, or other states or conditions of the subject. Determining the state or condition of a lung may comprise a diagnosis or determination of pneumonia, plural effusion, pulmonary embolism, poor airflow, chronic obstructive pulmonary disease, etc. The ECG, intrathoracic impedance, and/or audio data may detect the presence of fluid, crackles or gurgles in the lung. The neural network may compare the lung sounds and intrathoracic impedance measurements to diseased and healthy conditions of example lungs. Determining the state or condition of the subject may comprise indicating a presence or an increased level of a fluid in the lung of the subject. For example, intrathoracic impedance data measured by an intrathoracic sensor of the monitoring device may comprise information about a fluid in the lung of the subject.

Determining the state or condition of the subject may comprise conditions such as a presence or absence of atrial fibrillation, information about sinus rhythm such as normal or abnormal sinus rhythm, trigeminy, bigeminy, premature ventricular contraction, premature atrial contraction, or other conditions.

Determining the state of condition of a bowel comprises a diagnosis or determination of inflammatory bowel disease, intestinal obstruction, hernia, infection within the digestive tract, etc. The output may provide an indication of gastric motility or bowel function.

The state or condition of an organ of the subject may be determined at an accuracy of at least about 80%, 85%, 90%, 95%, 98%, 99%, or more for independent subjects. For example, the state or condition of the heart of the subject may be determined at an accuracy of at least about 80%, 85%, 90%, 95%, 98%, 99%, or more. For example, the state or condition of the lung of the subject may be determined at an accuracy of at least about 80%, 85%, 90%, 95%, 98%, 99%, or more. For example, the state or condition of the bowel of the subject may be determined at an accuracy of at least about 80%, 85%, 90%, 95%, 98%, 99%, or more. The state or condition of the subject may comprise an output of a trained algorithm, such as a neural network.

The state or condition of the organ of the subject may be determined at a specificity of at least about 80%, 85%, 90%, 95%, 98%, 99%, or more. The state or condition of the organ of the subject may be determined at a sensitivity of at least about 80%, 85%, 90%, 95%, 98%, 99%, or more. The state or condition of the organ of the subject may be determined at a specificity of at least about 80%, 85%, 90%, 95%, 98%, 99%, or more, and a sensitivity of at least about 80%, 85%, 90%, 95%, 98%, 99%, or more. The state or condition of the organ of the subject may be determined at a positive predictive value of at least about 80%, 85%, 90%, 95%, 98%, 99%, or more. The state or condition of the organ of the subject may be determined at a negative predictive value of at least about 80%, 85%, 90%, 95%, 98%, 99%, or more. The state or condition of the organ of the subject may be determined at an accuracy of at least about 80%, 85%, 90%, 95%, 98%, 99%, or more. The state or condition of the organ of the subject may be determined with an area under the receiver operating characteristic (AUROC) of at least about 0.75, 0.80, 0.85, 0.90, 0.95, 0.98, 0.99, or more.

The state or condition of an organ of the subject may be determined to be a no-failure state or condition at a specificity of at least about 80%, 85%, 90%, 95%, 98%, 99%, or more for independent subjects. For example, the state or condition of a heat of the subject may be determined to be a no-failure state or condition at a specificity of at least about 80%, 85%, 90%, 95%, 98%, 99%, or more for independent subjects. For example, the state or condition of a lung of the subject may be determined to be a no-failure state or condition at a specificity of at least about 80%, 85%, 90%, 95%, 98%, 99%, or more for independent subjects. The state or condition of a bowel of the subject may be determined to be a no-failure state or condition at a specificity of at least about 80%, 85%, 90%, 95%, 98%, 99%, or more for independent subjects. The state or condition of the heart in relation to heart murmurs such as mitral regurgitation (MR), or tricuspid regurgitation may be detected with greater than about 95% sensitivity and specificity. The state or condition of the heart in relation to atrial fibrillation may be detected with greater than 99% sensitivity and specificity. The state or condition of the heart in relation to congestive heart failure or heart failure may be detected with greater than 95% sensitivity and specificity.

The trained algorithm may be configured to identify the state or condition at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the state or condition by the trained algorithm may be calculated as the percentage of independent test samples (e.g., subjects known to have the state or condition or subjects with negative clinical test results for the state or condition) that are correctly identified or classified as having or not having the state or condition.

The trained algorithm may be configured to identify the state or condition with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the state or condition using the trained algorithm may be calculated as the percentage of datasets of ECG data and/or audio data identified or classified as having the state or condition that correspond to subjects that truly have the state or condition.

The trained algorithm may be configured to identify the state or condition with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the state or condition using the trained algorithm may be calculated as the percentage of datasets of ECG data and/or audio data identified or classified as not having the state or condition that correspond to subjects that truly do not have the state or condition.

The trained algorithm may be configured to identify the state or condition with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the state or condition using the trained algorithm may be calculated as the percentage of independent test samples associated with presence of the state or condition (e.g., subjects known to have the state or condition) that are correctly identified or classified as having the state or condition.

The trained algorithm may be configured to identify the state or condition with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the state or condition using the trained algorithm may be calculated as the percentage of independent test samples associated with absence of the state or condition (e.g., subjects with negative clinical test results for the state or condition) that are correctly identified or classified as not having the state or condition.

The trained algorithm may be configured to identify the state or condition with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operating Characteristic (ROC) curve (e.g., the area under the ROC curve) associated with the trained algorithm in classifying datasets of ECG data and/or audio data as having or not having the state or condition.

The trained algorithm may be adjusted or tuned to improve one or more of the performance, accuracy, PPV, NPV, clinical sensitivity, clinical specificity, or AUC of identifying the state or condition. The trained algorithm may be adjusted or tuned by adjusting parameters of the trained algorithm (e.g., a set of cutoff values used to classify a dataset of ECG data and/or audio data as described elsewhere herein, or parameters or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of features (e.g., of the ECG data, intrathoracic impedance data, and/or audio data) may be identified as most influential or most important to be included for making high-quality classifications or identifications of the state or condition. The plurality of features or a subset thereof may be ranked based on classification metrics indicative of each feature's influence or importance toward making high-quality classifications or identifications of the state or condition. Such metrics may be used to reduce, in some examples significantly, the number of input variables (e.g., predictor variables) that may be used to train the trained algorithm to a desired performance level (e.g., based on a desired minimum accuracy, PPV, NPV, clinical sensitivity, clinical specificity, AUC, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

The state or condition of the heart of the subject may be a type of a heart murmur, such as aortic stenosis (AS). Aortic stenosis may be a common disease which may be detected as a murmur on auscultation. A common method for detecting AS may be transthoracic echocardiography (TTE). In some examples, a referral from a healthcare provider who may have recognized an abnormality on auscultation may be needed for performing transthoracic echocardiography on subjects. However, in some examples, AS conditions may be hard to detect by physicians, particularly less experienced primary care physicians. For example, present AS conditions may be often not detected by less experienced primary care physicians. The methods of the present disclosure may facilitate the detection of AS at a sensitivity of at least about 80%, 85%, 90%, 95%, 98%, 99%, or more, and/or at a specificity of at least about 80%, 85%, 90%, 95%, 98%, 99%, or more. Furthermore, the methods may help quickly confirm suspected AS at a sensitivity of at least about 80%, 85%, 90%, 95%, or more, such as, for example, 97.2%, and a specificity of at least about 80%, 85%, 90%, 95%, or more, such as, for example, 86.4%. The state or condition of the subject may be determined using the trained algorithm. The trained algorithm may be trained for specific applications. For example, the trained algorithm may be trained for detection of aortic stenosis in which case it may be referred to as an Aortic Stenosis (AS) algorithm. The methods, devices and systems of the present disclosure may be used by healthcare providers during primary care visits. The methods of the present disclosure may facilitate the automatic detection of clinically significant AS, which may be further validated by transthoracic echocardiography (TTE). Phono- and electrocardiogram detection and analyses facilitated by the methods, devices and systems of the present disclosure may be used for detection of valvular and structural heart diseases.

The trained algorithm can also access a database to provide additional information that a healthcare provider may need to access or classify a state or condition of an organ of a subject. The database may comprise examples of ECG data and/or audio data of heartbeats associated with pre-existing certain states or conditions of the subject. The states or conditions can be related to a disease or healthy state, states or conditions comprising a biological or physiological condition, states or conditions comprising a diagnosis or determination, or unknown states. Further, the states or conditions can be related to an organ of the subject, such as, for example, a heart or a lung of the subject. The database may contain examples related to diagnoses or determinations of a low ejection fraction, normal ejection fraction, congestive heart failure, a heart failure risk score, arrhythmia, heart blockage, ischemia, infarction, pericarditis, hypertrophy, heart murmur, and more. For conditions like heart murmur, examples in the database may comprise diagnoses or determinations of a certain type of a heart murmur such as of a systolic murmur or a diastolic murmur. Moreover, examples in the database may comprise diagnoses or determinations of other conditions or states such as an aortic stenosis, a pulmonic stenosis, a mitral regurgitation, a tricuspid regurgitation, or a mitral valve prolapse, aortic regurgitation, a pulmonic regurgitation, a mitral stenosis, or a tricuspid stenosis, and more. The examples in the database can also include a healthcare provider's annotations on the determination of the state or condition of the subject, such as a diagnosis of a subject (e.g., a patient) in each case.

The trained algorithm may use the database to assist healthcare providers to identify or classify a state or condition of a subject based on the recorded audio data, ECG data, or a combination of audio and ECG data. The trained algorithm may compare the recorded audio data and ECG data associated with a condition or state separately or together in the database with recorded audio and/or ECG data using the disclosed sensor herein. For example, the algorithm may identify a number of examples from the database that are closest in terms of a plurality of features of ECG data and/or audio data to a recorded ECG or audio data of a subject using the sensor disclosed herein. Certain identified examples from the database may have similar intensity, pitch, or shape of recorded audio frequency data compared to recorded audio data by the monitoring device disclosed herein. Further, these identified examples from the database may have a similar average number of PR segments, ST segments, PR intervals, QRS intervals, ST intervals, or QT intervals of their ECG data compared to the ECG data recorded by the monitoring device disclosed herein. In some examples, the number of examples can be at least 1, 2, 3, 4, 5, 6, 7, 8, 9, 10, 11, 12, 13, 14, 15, 16, 17, 18, 19, 20, or more. In other examples, the number of examples can be 5. The number of examples is not meant to be limiting. The algorithm can search and locate several, such as 3, 4, 5, or 6 examples of recorded ECG and audio data associated with a certain type of heart murmur that contain the closest features compared to features associated with a ECG and/or audio data recorded by the disclosed sensor herein. This feature may be referred to as the k-nearest neighbor or n-nearest neighbor feature, where k or n may represent the number of examples identified from the saved database.

Subsequently, the algorithm can send a comparison of the closest examples from the database with the sensor generated ECG and/or audio data to a computing device or a cloud storage so a health care provider can have access to the comparison. The comparison may be sent to the computing device or cloud storage in real-time or substantially in real-time. This may facilitate decision-making regarding detecting or classifying a state or condition by taking into account relevant information about the subject and similarity of the recorded audio and ECG signals to examples from the database.

The trained algorithm provided in the present disclosure may provide healthcare providers with tools to more accurately detect states or conditions of subject, such as structural heart disease, during primary care visits.

### Computing Device

The present disclosure provides computing devices which may receive data from a monitoring device comprising sensors of varying modalities described elsewhere herein. For example, the computing device may receive ECG data, audio data, or other types of data recorded/captured by different types of sensors as disclosed elsewhere herein. In some examples, the computing device may be the same as the monitoring device, which may include one or more sensors. The computing device may comprise computer control systems that are programmed to implement methods of the disclosure.

The computing device may be configured to communicate with a monitoring device (e.g., the monitoring device 100 shown in FIGS. 1A and 1B). The computing device may communicate with the monitoring device through a wireless communication interface. As an alternative, the computing device may communicate with the monitoring device through a physical (e.g., wired) communication interface. The computing device may communication with the monitoring device through a wide area network (WAN) which may include the Internet. The computing device may communicate with the monitoring device through a cellular network. The computing device may communicate with the monitoring device through an infrared communication link. The computing device may be configured to communicate with the monitoring device via a radio-frequency communication. For example, the radiofrequency communication may be Bluetooth, may be a standard wireless transmission protocol (e.g., Wi-Fi), etc. The computing device may communicate with a server as part of a distributed computing system.

The computing device may be mobile. The computing device may be capable of movement from one place to another. The computing device may be a personal computer (e.g., portable PC, laptop PC), slate or tablet PC (e.g., Apple® iPad, Samsung® Galaxy Tab), telephone, Smart phone (e.g., Apple® iPhone, Android-enabled device, Blackberry®), or personal digital assistant.

The computing device may be separated from the monitoring device by a distance. For example, the distance may be within about 1 foot, 2 feet, 3 feet, 4 feet, 5 feet, 10 feet, 20 feet, 30 feet, 40 feet, 50 feet, 100 feet, 200 feet, 300 feet, 500 feet, 100 yards, 200 yards, 300 yards, 400 yards, 500 yards, 1000 yards, 1 mile, 5 miles, 10 miles, 100 miles, 500 miles, 1000 miles, 10,000 miles, 15,000 miles, or more between the monitoring device and the computing device.

In an example, the computing device may comprise a distributed computing system. In some examples, the distributed computing system may be in contact with a monitoring device and in connection with a mobile device. The computing device can be operatively coupled to a computer network (“network”). The network can be the Internet, an internet and/or extranet, or an intranet and/or extranet that is in communication with the Internet. The network in some examples is a telecommunication and/or data network. The network can include one or more computer servers, which can enable distributed computing, such as cloud computing. The network, in some examples with the aid of the computer system, can implement a peer-to-peer network, which may enable devices coupled to the computer system to behave as a client or a server.

The cloud computing network may enable remote monitoring of a subject. The cloud computing network may store subject data over time, such as ECG data, intrathoracic impedance data, and audio data. Subject data such as ECG data, intrathoracic impedance data, and audio data may be analyzed on a remote server via a cloud computing network. The remote server may perform calculations (such as analyzing data) with greater computational cost that a mobile device of a user. Alternatively, in some examples, the monitoring device may store the data (e.g., ECG data, audio data, or any other data) in internal memory.

The computing device, such as mobile device or a remote computing device may include a user interface. The ECG data and audio data or other data from any sensor or sensor modality provided herein may be transmitted to the computing device for display on the user interface. The data, or an output generated from such data, may be presented on the user interface over the time period in real-time or substantially real-time (e.g., a time delay of at most 1 millisecond with respect to when the data, such as the ECG data and audio data, was collected). In an example, the user interface is as a graphical user interface. Examples of user interfaces include, without limitation, a graphical user interface (GUI), web-based user interface, a mobile user interface, an app, etc. The user interface may comprise an app (e.g., a mobile application) as described elsewhere herein.

The user interface may comprise a web-based interface. For example, the web-based interface may be a secure web browser. The web-based interface may be a secure web page. The universal resource locator (URL) of the secure web page may be changed at the request of a user. Access data on the secure web page may be protected by a password. The URL may comprise a unique token which is generated for each session. The unique token may be given to a subject and/or a third party. The token may be associated with a subject. In some examples, the token may be associated with a session. The token may be associated with a third-party operator such as a physician. The token may comprise two-factor identification. The token may rotate with time. The token may be reissued or reassigned at any time. The secure web browser may be encrypted. The token may be associated with a cryptographic key. The token may be associated with biometric data. The token may be a single sign-on token.

In some examples, after transmitting and processing the data from the monitoring device (e.g., ECG data, intrathoracic impedance data, and audio data) to the computing device, the processed data (e.g., ECG data, intrathoracic impedance data, and audio data) indicating a state or condition of an organ of a subject can be transmitted back to the monitoring device. The monitoring device may be synced in real-time or substantially real-time with the computing device such as a mobile device. The transmission of processed data (e.g., ECG data, intrathoracic impedance data, and audio data) from the computing device to the monitoring device is in real-time or substantially real-time. An output indicative of the determined state or condition of the subject may be provided on the monitoring device through an audio broadcasting so that a healthcare provider can hear the output in real-time or substantially real-time. Further, the output may include an intervention/treatment plan based on the determined state or condition of the subject, follow-up tests, preventive plans, and/or pharmaceuticals.

FIG. 7 shows a computer system (also referred to herein as a “computing device”) 701 that is programmed or otherwise configured to receive ECG data, intrathoracic impedance data, accelerometer (e.g., motion and orientation) data, and audio data from a monitoring device (e.g., the monitoring device 100 shown in FIGS. 1A and 1B). The computer system 701 can regulate various aspects of the monitoring device of the present disclosure, such as, for example, processing the ECG, intrathoracic impedance, accelerometer, and/or audio data, providing an output indicative of a state or condition of a subject, and providing a log of data over time. In some embodiments, the computer system 701 may be a computing device of a user or a computer system that is remotely located with respect to the monitoring device. The computing device can be a mobile computing device.

The computer system 701 includes a central processing unit (CPU, also “processor” and “computer processor” herein) 705, which may be a single core or multi core processor, or a plurality of processors for parallel processing. The computer system 701 also includes a memory or memory location 710 (e.g., random-access memory, read-only memory, flash memory), an electronic storage unit 715 (e.g., hard disk), a communication interface 720 (e.g., network adapter) for communicating with one or more other systems, and peripheral devices 725, such as cache, other memory, data storage, and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 530 with the aid of the communication interface 720. The network 530 can be the Internet, an internet and/or extranet, or an intranet and/or extranet that is in communication with the Internet. The network 530 in some examples is a telecommunication and/or data network. The network 530 can include one or more computer servers, which can enable distributed computing, such as cloud computing. The network 530, in some examples with the aid of the computer system 701, can implement a peer-to-peer network, which may enable devices coupled to the computer system 701 to behave as a client or a server. The computer system 701 can include or be in communication with an electronic display 735 that comprises a user interface (UI) 740 for providing, for example, an output indicative a state or condition of a user.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and write back.

The CPU 705 can be part of a circuit, such as an integrated circuit. One or more other components of the system 701 can be included in the circuit. In some examples, the circuit is an application specific integrated circuit (ASIC).

The computing device may store ECG data and audio data. The computing device may store ECG data and audio data on a storage unit. The storage unit 715 can store files, such as drivers, libraries and saved programs. The storage unit 715 can store user data, e.g., user preferences and user programs. The computer system 701 in some examples can include one or more additional data storage units that are external to the computer system 701, such as located on a remote server that is in communication with the computer system 701 through an intranet or the Internet.

The computer system 701 can communicate with one or more remote computer systems through the network 530. For instance, the computer system 701 can communicate with a monitoring device. In some embodiments, the computing device is a remote computer system. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. In some examples, the user can access the computer system 701 via the network 530.

Methods as described herein can be implemented by way of machine (e.g., computer processor) executable code stored on an electronic storage location of the computer system 701, such as, for example, on the memory 710 or electronic storage unit 715. The machine executable or machine-readable code can be provided in the form of software. During use, the code can be executed by the processor 705. In some examples, the code can be retrieved from the storage unit 715 and stored on the memory 710 for ready access by the processor 705. In some examples, the electronic storage unit 715 can be precluded, and machine-executable instructions are stored on memory 710.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables, copper wire, and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Mobile Application

Referring now to FIG. 13, a mobile application workflow 1300 is provided herein. A mobile application may provide the capability to initiate data collection, to stop data collection, to store data, to analyze data, and/or to communicate with a remote server or distributed computing network. In an example, the mobile application is installed on the mobile device of a user, such as a subject. In another example, the mobile application may be accessed by a web browser. The mobile application and the web-based interface may comprise substantially similar functionality.

In an example, a user may initiate a software application installed on a mobile device 1302, such as a smart phone or a laptop. In some examples, the mobile application is downloaded by a user. The mobile application may comprise instructions such as machine-readable code which may be executed by a processor, such as a central processing unit (CPU) or a micro-processing unit (MPU), of the present disclosure. When executed, the instructions may control the operation of the monitoring device 100 first introduced in FIGS. 1A and 1B. The mobile application may comprise a user interface, as described elsewhere herein. The user interface may provide guidance and instructions to the user via the user interface. For example, the mobile application may provide visual displays on a display screen to illustrate proper placement of the monitoring device on the body of the subject.

The subject or another user may place the monitoring device 100 on the subject's skin. The mobile device may provide guidance as to proper placement. The electrodes 110A and 110B (shown in FIG. 1A) may contact the skin of the subject. The electrodes 110A and 110B may measure electrical changes on the skin and or sound created from a patient organ.

The subject or another user may press the button 120 to initiate monitoring of the organ of the subject. Depression of the button 120 may initiate simultaneous recording from multiple sensor modalities. The subject may hold the monitoring device 100 against their own chest, or another user may hold the monitoring device 100 against the subject's chest. In some examples, the button 120 remains depressed in order to take subject data. In other examples, a first press of the button 120 starts collection and a second press of the button 120 stops collection. In other examples, data collection may be stopped and started by a web-based interface.

After the button 120 is depressed, patient data may be collected, such as ECG data, intrathoracic impedance data, accelerometer data, and audio data. The collected data may be pre-processed on the monitoring device 100. For example, pre-processing may comprise amplification, filtering, compression, etc. of the data. In some examples, the data may be stored locally for a time. The collected data, which may comprise pre-processed data, may then be transmitted to the mobile device 1302 (or other computing device). The collected data may be transmitted to the mobile device 1302 in real-time. The collected data may be displayed on a user interface 1304 of the mobile device 1302 in substantially real-time. The transmitted data may be accessed via a mobile application on the mobile device 1302. In some examples, the transmitted data may also be accessible via a web-based interface.

The data collected from the organ of the subject may be published to other computing devices of other users involved in the subject's care. For example, the ECG, intrathoracic impedance, accelerometer, and audio data may be transmitted to a server via a network. The server may store subject data long term. The server may analyze data that may require greater processing power than may be possible on the mobile device 1302; however, in some embodiments, the data may be analyzed on the mobile device 1302. In some examples, the server may be accessible by the computing device of a health care provider. The web-based interface may additionally be accessible by the computing device of a health care provider. The subject may use the monitoring device 100 at home or in a remote location and may make data from the monitoring device 100 available to a health care provider. The data available to a healthcare provider may enable remote diagnosis.

The data from the monitoring device may be available to third parties in real-time or substantially real-time. The data may be stored over time within the server. The data collected over multiple time periods may be stored within the server. The server may comprise a repository of historical data for later analysis.

The mobile application may provide feedback to the subject or to a user on the quality of the data. For example, a voice-based audio feedback may alert the subject. The mobile application may use a speaker of the mobile device 1302. In another example, an on-screen alert may be visually displayed to alert the subject via the user interface 1304. The subject may be alerted during the course of acquisition of ECG, intrathoracic impedance, and audio data. The monitoring device 100 may execute a local application on the MPU to alert a user on the monitoring device 100. The mobile device 1302 may execute a local application on the CPU to alert a user on the mobile application. The mobile application may display an alert and play audio feedback simultaneously.

The mobile application may additionally display instructions to increase data quality, such a instructing the subject or a user to change a position of the monitoring device 100. The mobile application may instruct the patient to stay still, such as when the accelerometer 150 (shown in FIG. 1A) detects motion. The mobile application may alert a subject when data collection is complete. The mobile application may alert a subject when data quality is poor. The mobile application may display previous results. The mobile application may prompt a user to start a new data acquisition session. The mobile application may alert the subject when data has been reviewed by a health care provider. A health care provide may comprise a clinician, a physician, and/or another trained operator.

The mobile application may display a waveform of subject ECG data. The mobile application may display a waveform of subject audio data. The subject may simultaneously view both waveforms. The subject may view the waveforms in real-time. A remote user may view one or both waveforms from a remote location in real or substantially real-time. The user may be able to compare differences or similarities between the data. The user may be able to spot issues in collecting the data, such as waveform irregularities from excess movement, speaking, poor sensor contact, etc. The user may be able to monitor his or her own heartrate.

As an illustrative example, the mobile application may include or communicate with an analysis software 1308 that may analyze simultaneous ECG and heart audio data to detect the presence of suspected murmurs in the heart audio data. The analysis software 1308 may also detect the presence of atrial fibrillation and normal sinus rhythm from the ECG signal. In addition, the analysis software 1308 may calculate certain cardiac time intervals such as heart rate, QRS duration, and EMAT. In the present example, the analysis software 1308 is a cloud-based software application programming interface (API) that allows a user to upload synchronized ECG and heart audio or phonocardiogram (PCG) data for analysis. The analysis software 1308 uses various methods to interpret the acquired signals, including signal processing and artificial neural networks. The API may be electronically interfaced and may perform analysis with data transferred from multiple mobile-based or computer-based applications.

The analysis software 1308 is configured to be used in conjunction with a system of the present disclosure (e.g., comprising one or more of ECG sensors, audio sensors, force sensors, vibration sensors, temperature sensors, pressure sensors, respiratory monitors or sensors, heart rate monitors or sensors, intrathoracic impedance monitors or sensors, and/or other types of sensors), a companion mobile application (app) on the mobile device 1302, and a cloud-based infrastructure 1306. The system may be configured to capture heart audio only, or both heart audio and ECG data (e.g., a single-lead ECG). The heart audio and ECG signals are transmitted to the mobile app using Bluetooth Low Energy. When a user makes a recording via the monitoring device 100, a .WAV file is generated by the mobile app on the mobile device 1302 and transmitted to the cloud-based infrastructure 1306, where the .WAV file is saved. This also triggers the analysis software 1308 API to perform analysis of the .WAV file. The analysis software 1308 is configured to output a JSON file with the algorithm results, which is passed down to the mobile device 1302 and displayed using the same mobile app via the user interface 1304.

For example, as shown in FIG. 13, the monitoring device 100 may first perform a data transfer to the mobile device 1302 via a Bluetooth Low Energy protocol. Second, a .WAV file is uploaded from the mobile device 1302 to the cloud-based infrastructure 1306 (e.g., EkoCloud). Third, data from the cloud-based infrastructure 1306 is sent for analysis using the analysis software 1308, shown as an electronic analysis software (EAS) API. Fourth, the analysis results are returned from the EAS to the cloud-based infrastructure 1306 as a JSON file. Fifth, the analysis results are sent from the cloud-based infrastructure 1306 to the mobile device 1302 and displayed in a mobile app of the mobile device via the user interface 1304.

The analysis software 1308 comprises the following algorithms of the present disclosure: (1) a rhythm detection algorithm that uses a neural network model to process ECG data to detect normal sinus rhythm and atrial fibrillation; (2) a murmur detection algorithm that uses a neural network model to process heart audio data to detect the presence of murmurs; (3) a Heart Rate algorithm comprising a signal processing algorithm that processes ECG data or heart audio data to calculate the heart rate of a subject, and provides an alert if the measured heart rate is indicative of an arrhythmia such as bradycardia or tachycardia; (4) a QRS duration algorithm comprising a signal processing algorithm that processes ECG data to measure the width of the QRS pulse; and (5) an EMAT interval algorithm comprising a signal processing algorithm that uses Q peak detection and S1 envelope detection to measure the Q-S1 interval, defined as electromechanical activation time or EMAT.

The analysis software 1308 comprises signal quality algorithms to assess the quality of the incoming ECG and PCG data. The model determines whether the recording is of sufficient signal quality to run the classifier algorithms. The signal quality indicators were trained based on noise annotations and/or poor signal annotations from the training dataset. Those annotations indicated whether the signal quality was too poor to reliably classify arrhythmias or heart murmurs (from ECG and heart audio data, respectively). That training effort resulted in signal quality analysis algorithms that determine whether the data is of sufficient quality and, if it is not, labels the recording as “Poor Signal.” The signal quality algorithms are used prior to analysis by the algorithms described below. Additionally or alternatively, accelerometer data may be used to gate the incoming ECG and PCG data such that the analysis and detection algorithms described herein do not use ECG and PCG data generated during patient movement. For example, the signal quality indicator may label the recordings as “Device Motion” when the root mean square amplitude of acceleration is greater than a pre-defined threshold. The pre-defined threshold may be a non-zero root mean square amplitude of acceleration below which slight patient movements may not interfere with ECG and PCG data generation and the resulting analysis, as will be elaborated below with respect to FIG. 14.

The rhythm detection algorithm is configured to detect normal sinus rhythm and atrial fibrillation from ECG waveforms using a deep neural network model trained to classify ECGs into one of four categories: normal sinus rhythm, atrial fibrillation, unclassified, or poor signal. Following a determination of sufficient quality by the signal quality ECG algorithm, the rhythm detection classifier determines whether the signal shows presence of “Atrial Fibrillation” or can be classified as “Normal Sinus Rhythm” or represents other rhythms and is labeled as “Unclassified”.

The murmur detection algorithm is configured to detect heart murmurs using a deep neural network model trained to classify heart sound recordings as containing a heart murmur or containing no detectable (e.g., no audible) murmur. Following a determination of sufficient quality by the signal quality PCG algorithm, the murmur detection classifier decides whether the signal shows presence of a “murmur” or can be classified as “no murmur.”

An output indicative of the result may be communicated or conveyed to the user (e.g., subject or healthcare provider). The output may be communicated or conveyed in various forms, such as displaying a message on the monitoring device, computing device, or both, or any other device which may be configured to communicate with the monitoring and/or computing device (e.g., remotely, wirelessly, or otherwise). The output may be in the form of a display, audio/sound recording which may be communicated through the earpieces, haptic feedback, a written document of any format, such as a pdf or word document, or other forms. The output may be capable of and/or configured to be shared on a mobile device. For example, the output may be shared as a pdf file. Examples of the outputs communicated or otherwise provided to the user may comprise “no murmur detected,” “murmur detected,” “poor signal,” “device motion,” “poor signal quality,” “systolic murmur detected,” “diastolic murmur detected,” “flow murmur detected,” “aortic stenosis detected,” “mitral regurgitation detected,” “innocent murmur detected,” “still's murmur detected,” “mitral stenosis detected,” “aortic regurgitation detected,” “ventricular septal defect detected,” “atrial septal defect detected,” “pulmonic regurgitation detected,” “pulmonic stenosis detected,” “mitral stenosis detected,” “patent ductus arteriosus detected,” “holosystolic murmur detected,” “continuous murmur detected,” “crescendo-decrescendo murmur detected,” “systolic decrescendo murmur detected,” “diastolic decrescendo murmur detected,” or other outputs. The output may further comprise information about the severity grading of the state or condition, such as a heart murmur or other condition listed anywhere herein. For example, the output may comprise “mild,” “moderate,” or “severe.” The output may be indicative of any state or condition of the subject, such as a state or condition of an organ or organ system of the subject comprising heart, lungs, bowel, skin, or other body parts, such as the body parts and/or conditions provided anywhere herein. For example, an output may be indicative of the presence of a fluid in the lungs of the subject. In each example, a suitable output may be conveyed to the user(s) in a suitable way.

The heart rate algorithm is configured to determine a heart rate using a signal processing algorithm that uses ECG or heart audio data. If ECG data are present and are determined to be of sufficient signal quality, then the median R-R interval from the detected QRS complexes is used to calculate the heart rate. If ECG data are absent or of poor quality, the heart rate is computed from the PCG signal if it has good signal quality using an auto-correlation based analysis. If the signal quality of the PCG is also poor, then no heart rate value is presented. The ECG-based heart rate algorithm is a modified version of the classical Pan-Tompkins algorithm. In addition, EAS also generates a “Bradycardia” alert if the measured heart rate is below 50 BPM and a “Tachycardia” alert if the measured heart rate is above 100 BPM.

The EMAT algorithm comprises a signal processing algorithm configured to determine an EMAT. Following a determination of sufficient quality by the signal quality PCG and ECG algorithms, the EMAT algorithm uses Q peak detection on the ECG and S1 envelope detection on heart audio data to measure the Q-S1 interval, defined as electromechanical activation time or EMAT. EMAT interval calculation requires simultaneous recording of ECG and heart audio data. The reported % EMAT for an entire recording is reported as the median % EMAT of all beats in the signal.

The analysis software 1308 may be configured to interface with a user interface software API. The analysis software may be configured to receive data from and provide results to other software applications through an API. The API result can be displayed by any mobile app or web interface to the clinician without any modifications to the terms or result.

The analysis software 1308 may be used to aid medical professionals in analyzing heart sounds and ECG data captured from hardware devices. The analysis software may also be used to support medical initiatives in digital collection and analysis of physiological data to provide more efficient healthcare. For example, the adoption of electronic health records may facilitate the continuity of health care but must be augmented by other technologies to increase real-time access to patient data.

As a clinical evaluation method, auscultation may encounter challenges because of subjectivity, inability to quantify cardiovascular and pulmonary problems, and imprecision. For example, internal medicine and family practice trainees may accurately recognized only 20% of heart sounds. Heart audio analysis software can compensate for the limitations of acoustic stethoscopes. The analysis software 1308 is configured to detect the presence of murmurs in heart sounds, which then prompts the physician to conduct a more complete analysis of the detected murmur to determine whether it is innocent or pathologic. The analysis software's detection of the presence of murmurs are combined with clinician interpretations of heart sounds, of visualizations of heart sounds, and physician gestalt of clinical context to better determine appropriate follow-up. Although auscultation alone yields significant cardiac health information, synchronized ECGs can improve interpretation, as the data can provide insight into the heart rate and rhythm regularity. In addition, the analysis software 1308 is configured to perform atrial fibrillation detection using the single-lead ECG. The analysis software 1308 analyzes both ECG data and heart audio data to provide a comprehensive analysis of the electrical and mechanical function (as well as disorders) of the heart. For example, prolongation of the QRS duration can be indicative of a left ventricular dysfunction, such as left bundle branch block, which can be reported or conveyed to a user or a health care provider or anyone who is interested in receiving such output. The length of the QRS interval may be analyzed and the output may be displayed as a result of such analysis. Some additional conditions associated with the length of the QRS interval, such as “wide QRS complex” or “hyperkalemia” can be further displayed. Further, depending on the length of the QRS interval, a degress of hyperkalemia may be displayed via the output and communicated to a user, such as “mild hyperkalemia,” “moderate hyperkalemia,” or “severe hyperkalemia.”

The analysis software algorithms were validated using retrospective analysis on a combination of publicly available (MIT-BIH Arrhythmia Database, MIT-BIH Arrhythmia Noise Stress Database, Physionet QT Database, and PhysioNet 2016 Database) and other databases. The recordings used for validation were distinct from data sets used to train the algorithm. As summarized in the below tables, each of the algorithms exhibited excellent performance in performing their respective detection tasks.

The algorithm's performance for rhythm detection is summarized in Tables 1A and 1B. These results show that the algorithm accurately identifies when the hardware gives a usable and good ECG signal. When a good signal is detected, the algorithm detects Atrial Fibrillation and Normal Sinus Rhythm with high accuracy (with a sensitivity and a specificity greater than the minimal clinical requirement of 90% sensitivity and 90% specificity).

TABLE 1A
Rhythm detection on an ECG database (cases with good signal)

| Performance | Prevalence (%) | Sensitivity (%) |
|---|---|---|
| Good Signal | 74.6% | 85.7% |
| | (95% CI: 71.3%-77.6%) | (95% CI: 82.7%-88.2%) |

TABLE 1B
Rhythm detection on an ECG database (cases
with atrial fibrillation detection)

| Performance | Sensitivity (%) | Specificity (%) |
|---|---|---|
| Atrial | 100.0% | 96.0% |
| Fibrillation | (95% CI: 93.4%-100.0%) | (95% CI: 93.5%-97.6%) |
| Detection | | |

The algorithm's performance for murmur detection is summarized in Tables 2A and 2B. These results show that the algorithm accurately identifies when the hardware gives a usable and good heart sound. Further, the algorithm detects the presence of murmur with high accuracy (with a sensitivity and a specificity greater than the minimal clinical requirement of 80% sensitivity and 80% specificity).

TABLE 2A
Murmur detection on a heart sound
database (cases with good signal)

| Performance | Prevalence (%) | Sensitivity (%) |
|---|---|---|
| Good Signal | 87.8% | 94.8% |
| | (95% CI: 86.0%-89.4%) | (95% CI: 93.5%-95.9%) |

TABLE 2B
Murmur detection on a heart sound database
(cases with murmur detection)

| Performance | Sensitivity (%) | Specificity (%) |
|---|---|---|
| Murmur | 87.6% | 87.8% |
| Detection | (95% CI: 84.2%-90.5%) | (95% CI: 85.3%-89.9%) |

The algorithm's performance for murmur detection is summarized in Tables 3A and 3B. These results show that the algorithm calculates heart rate with an error of less than the clinically acceptable limit of 5%. Further, the algorithm can accurately detect the presence of bradycardia and tachycardia (with a sensitivity and a specificity greater than the minimal clinical requirement of 90% sensitivity and 90% specificity) and generate alerts for a clinician accordingly.

TABLE 3A
Heart rate detection on the MIT-BIH database (heart rate error)

| Performance | ECG |
|---|---|
| Heart Rate error (%) | 1.16% |
| | (95% CI: 0.96%-1.36%) |

TABLE 3B
Heart rate detection on the MIT-BIH database
(cases with bradycardia or tachycardia)

| Performance | Sensitivity (%) | Specificity (%) |
|---|---|---|
| Bradycardia | 98.0% | 97.6% |
| | (95% CI: 94.3%-99.3%) | (95% CI: 97.2%-98.1%) |
| Tachycardia | 94.6% | 98.3% |
| | (95% CI: 91.8%-96.5%) | (95% CI: 97.9%-98.7%) |

The algorithm's performance for QRS duration detection is summarized in Table 4. These results show that the algorithm can calculate the QRS duration with an error of less than the clinically acceptable limit of 12%.

TABLE 4
QRS duration detection on the PhysioNet QT database

| Performance | Mean | Standard Dev |
|---|---|---|
| Absolute QRS | 10.1 | 7.64 |
| error (ms) | (95% CI: 8.55-11.6) | (95% CI: 6.70-8.91) |
| Relative QRS | 9.20% | 6.11% |
| error (%) | (95% CI: 7.98%-10.4%) | (95% CI: 5.35%-7.12%) |

The algorithm's performance for EMAT duration detection is summarized in Table 5. These results show that the algorithm can calculate the EMAT duration with an error of less than the clinically acceptable limit of 5% of the average R-R interval.

TABLE 5
EMAT detection on an ECG database

| Performance | Actual |
|---|---|
| Absolute EMAT error (%) | 1.43% |
| | (95% CI: 1.15%-1.70%) |

In another example, a machine learning algorithm is developed to perform diabetic flow monitoring of a fluid status (e.g., blood). Patients with diabetes (e.g., type I or type II) may have a need to maintain a desired fluid volume, since their bodies may be unable to remove fluid as effectively as needed. However, conventional approaches of monitoring fluid volume or fluid flow may require invasive approaches involving venipuncture. Using systems and methods of the present disclosure, audio data of a fluid circulation of a subject may be collected and analyzed to determine a property of a fluid (e.g., blood) in the subject's body, such process may be used to replace the conventional venous access procedures, such as peripherally-inserted central catheters (PICC). This collection and analysis of audio data may be performed non-invasively with ECG sensors and/or audio sensors, without the use of venipuncture. The audio data of the fluid circulation may comprise audio data of blood flow across a fistula (e.g., a diabetic fistula) of the subject. The property of the fluid may comprise, for example, a fluid flow (e.g., a flow rate indicative of a volume of fluid per unit time), a fluid volume, a fluid blockage, or a combination thereof, of the subject. The property of the fluid may be characteristic of the fluid in a localized area of the subject's body, such as a location of vascular access or a diabetic fistula of the subject. One or more properties of the fluid, such as a fluid flow (e.g., a flow rate indicative of a volume of fluid per unit time), a fluid volume, or a fluid blockage, may be identified, predicted, calculated, estimated, or inferred based on one or more other properties of the fluid. For example, a flow volume (e.g., of blood) may be calculated or estimated based on a determined flow rate of the fluid.

Using systems and methods of the present disclosure, ECG data and/or audio data are collected from a plurality of different locations or parts of a body (e.g., organs or organ systems) of a subject, and then aggregated to provide an aggregate quantitative measure (e.g., a sum, an average, a median) of the plurality of different locations or parts of the body of the subject. The aggregate quantitative measure is then analyzed to determine a state or condition of the subject.

In some embodiments, the ECG data and/or audio data are collected from the plurality of different locations or parts of the body of the subject by a plurality of ECG sensors or leads (e.g., a 3-lead, 6-lead, or 12-lead ECG sensor) and/or audio sensors located at each of the plurality of different locations or parts of the body of the subject. In some embodiments, the ECG data and/or audio data are collected from the plurality of different locations or parts of the body of the subject by moving the ECG sensor and/or audio sensor to each of the plurality of different locations or parts of the body of the subject. The movement of the sensors may be performed by the subject or by a health provider (e.g., physician, nurse, or caretaker) of the subject.

In some embodiments, the ECG data comprise QT intervals, which may be analyzed to detect long QT intervals of the subject (which may correlate with or be indicative of an increased risk of heart failure of the subject). The QT interval measurements may be obtained by averaging ECG data acquired from a plurality of different locations of the heart of the subject. In some embodiments, a system or device of the present disclosure may comprise a sensor (e.g., an accelerometer) configured to detect if the device has been moved to different positions of the body (e.g., different positions of the heart) of the subject. The system or device may be configured to collect and analyze information of one or more movements or locations of the ECG sensor and/or the audio sensor corresponding to at least a portion of the ECG data and/or the audio data.

Turning now to FIG. 14, a flow chart of an example method 1400 for utilizing data from an accelerometer of a monitoring device to gate processing of physiological data obtained by the monitoring device is shown. The monitoring device may be the monitoring device 100 introduced in FIGS. 1A and 1B, for example, and may include an ECG sensor and an audio sensor to record the physiological data from a subject. Instructions for carrying out the method 1400 may be executed by one or more processors, such as the CPU 705 shown in FIG. 7, based on instructions stored on a memory of each of the one or more processors and in conjunction with signals received from the monitoring device. Although the method 1400 will be described with respect to processing ECG data and audio data, the method 1400 may be applied to processing other data types without departing from the scope of this disclosure.

At 1402, the method 1400 includes receiving ECG data, audio data, and acceleration data from the monitoring device in real-time. For example, the monitoring device may record the ECG data via the ECG sensor (e.g., an electrical sensor), record the audio data via the audio sensor, and record the acceleration data via the accelerometer and transmit the recorded data to the one or more processors via a wireless connection, such as a BLE connection, in real-time. The ECG data, the audio data, and the acceleration data may be time-aligned, such that the ECG data, the audio data, and the acceleration data may comprise data obtained over a common time period. In some examples, the ECG data, the audio data, and the acceleration data may be transmitted via a common data packet, such as the data packet structure shown in FIG. 6.

At 1404, the method 1400 includes determining a motion of the monitoring device based on the acceleration data. For example, the motion may be computed by integrating the acceleration with respect to each of the three axes of the accelerometer. Thus, the motion may be a velocity of the monitoring device.

At 1406, the method 1400 includes determining if the motion is greater than a motion threshold. The motion threshold may be pre-determined a non-zero motion value (e.g., velocity value) stored in memory that distinguishes smaller movements that will not affect analysis of the ECG data and the audio data from larger movements that may produce an inaccurate analysis or motion artifacts. As another example, the motion threshold may be a threshold root mean square amplitude of acceleration, and it may be determined that the motion is greater than the motion threshold when a root mean square amplitude of the acceleration measured by the accelerometer is greater than the threshold root mean square amplitude of acceleration.

If the motion is not greater than the motion threshold (e.g., the motion is less than or equal to the motion threshold), the method 1400 proceeds to 1408 and includes processing the ECG data and the audio data via an analysis algorithm. For example, the one or more processors may use any or all of the algorithms described herein for determining a state or condition of the subject. As another example, processing the ECG data and the audio data may include outputting a visual representation of the ECG data and the audio data on a user interface or other display, such as the user interface 1304 shown in FIG. 13.

In the example shown in FIG. 14, processing the ECG data and the audio data via the analysis algorithm includes determining an orientation of the monitoring device from the acceleration data, as indicated at 1410. In particular, the orientation may only be computed while the monitoring device is not moving. For example, the one or more processors may determine an angle of the monitoring device in each of the three axes of the accelerometer with respect to a three-dimensional world coordinate frame based on acceleration due to gravity measured in each of the three axes. Such a calculation may not be performed while the device is in motion (e.g., while the motion is greater than the motion threshold). Thus, the acceleration data from the accelerometer advantageously enables two different parameters to be determined, the motion of the monitoring device and the orientation of the monitoring device, during different monitoring device states (in motion or stationary, respectively).

Processing the ECG data and the audio data further includes determining an ECG vector based on a shape of the ECG data and the determined orientation of the monitoring device, as indicated at 1412. For example, the analysis algorithm may construct an ECG waveform from the ECG data and further analyze the ECG waveform in combination with the determined orientation to determine the ECG vector. The ECG vector may be further used by the analysis algorithm in determining the state or condition of the subject, as different ECG vectors may have different diagnostic capabilities. For example, some ECG vectors be more or less informative for identifying particular rhythmic and ischemic abnormalities than others.

The method 1400 may then end. For example, the method 1400 may be repeated at a pre-determined frequency so that the real-time ECG data and audio data will continue to be processed while the motion remains less than or equal to the motion threshold.

Returning to 1406, if the motion is greater than motion threshold, the method 1400 proceeds to 1414 and includes not processing the ECG data and the audio data via the analysis algorithm. For example, the one or more processors may omit the ECG data and the audio data obtained while the monitoring device is in motion from being used for determining the state or condition of the subject by the analysis algorithm. Further, because the monitoring device is in motion, the orientation of the monitoring device cannot be determined. Hence, the one or more processors may not determine the ECG vector. The method 1400 may then end. For example, the method 1400 may be repeated so that the ECG and audio data may be processed once the monitoring device is no longer in motion, as determined by the motion decreasing below the motion threshold, for example. By not processing the ECG data and the audio data in response to device motion, an accuracy of the analysis may be increased.

Next, FIG. 15 shows a flow chart of an example method 1500 for utilizing data from an accelerometer of a monitoring device to adjust an audio gain of audio data recorded by the monitoring device and transmitted to a listening device. The monitoring device may be the monitoring device 100 introduced in FIGS. 1A and 1B, for example, and may include an audio sensor to record physiological sounds from a subject. Instructions for carrying out the method 1500 may be executed by one or more processors, such as the MPU 505 shown in FIG. 5, based on instructions stored on a memory of each of the one or more processors and in conjunction with signals received from the sensors of the monitoring device. In some examples, the method 1500 may be performed concurrently and/or in combination with the method 1400 of FIG. 14.

At 1502, the method 1500 includes receiving the audio data and acceleration data in real-time. For example, the monitoring device may record the audio data via the audio sensor and record the acceleration data via the accelerometer, which may each transmit the recorded data to the one or more processors in real-time. In examples where the one or more processors are external to the monitoring device, the monitoring device may transmit the recorded data to the external processors via a wireless connection, such as a BLE connection, in real-time. The audio data and the acceleration data may be time-aligned, such that the audio data and the acceleration data may comprise data obtained over a common time period.

At 1504, the method 1500 includes determining a motion of the monitoring device based on the acceleration data. For example, the motion may be computed by integrating the acceleration with respect to each of the three axes of the accelerometer. Thus, the motion may be a velocity of the monitoring device.

At 1506, the method 1500 includes determining if the motion is greater than a motion threshold. The motion threshold may be pre-determined a non-zero motion value (e.g., velocity value) stored in memory that distinguishes smaller movements that will not produce motion artifacts, such as movement noises, from larger movements that may produce motion artifacts. As another example, the motion threshold may be a threshold root mean square amplitude of acceleration, and it may be determined that the motion is greater than the motion threshold when a root mean square amplitude of the acceleration measured by the accelerometer is greater than the threshold root mean square amplitude of acceleration.

If the motion is not greater than the motion threshold (e.g., the motion is less than or equal to the motion threshold), the method 1500 proceeds to 1508 and includes transmitting the audio data to the listening device with high audio gain. For example, a listener may listen to the audio data in real-time via the listening device. The listening device may be earpieces, such as the earpieces 204 shown in FIG. 2, or a speaker. The listening device may be connected to the monitoring device and/or the one or more processors via wired or wireless communication. Transmitting the audio data to the listening device with the high audio gain may enable the listener to hear quiet physiological sounds, such as heart sounds, lung sounds, or bowel sounds, as increasing the audio gain of the audio data input to the listening device increases an output volume of the listening device. Because device motion is not detected, motion artifacts will not be amplified. The method 1500 may then end. For example, the method 1500 may be repeated at a pre-determined frequency so the audio gain may be adjusted as the motion of the monitoring device changes.

Returning to 1506, if the motion is greater than motion threshold, the method 1500 proceeds to 1510 and includes reducing the audio gain of the audio data transmitted to the listening device. For example, the audio gain may be reduced responsive to the motion of the monitoring device being greater than the motion threshold so that motion artifacts recorded due to the device movement will not be amplified. Because amplifying the motion artifacts may result in loud, unpleasant noises being output to the listener via the listening device, reducing the audio gain in response to device motion may increase listener comfort. The method 1500 may then end. For example, the method 1500 may be repeated so that the audio gain may be increased once the monitoring device is no longer in motion, as determined by the motion decreasing below the motion threshold, for example. Thus, the audio data may be transmitted to the listening device with a first, higher gain responsive to the motion of the monitoring device not be greater than the threshold motion and may be transmitted to the listening device with a second, lower gain responsive to the motion of the monitoring device being greater than the threshold motion.

In this way, physiological data recorded by a monitoring device may be efficiently processed in real-time, and data processing may be adjusted based on a determined motion and/or a determined orientation of the motioning device. For example, analysis algorithms may strategically process data obtained while the device is stationary in order to reduce motion artifacts and increase an accuracy of the results. As another example, an audio gain may be reduced responsive to detected motion so that audio data projected by a listening device, such as a speaker, may not include amplified motion artifacts. The motion and orientation may both be determined based on signals received from an accelerometer of the monitoring device. For example, the motion may be determined, and the orientation may be subsequently determined responsive to the determined motion being less than a motion threshold. Further, the orientation data may be used to help determine a vector of ECG data recorded by the monitoring device. Further still, electrodes used to measure the ECG data also may be used to measure intrathoracic impedance data. By obtaining two types of physiological data with the same electrodes, a utility of the monitoring device may be increased while decreasing a cost and size of the monitoring device (e.g., compared with including separate sensors for measuring ECG data and intrathoracic impedance data).

The technical effect of using motion data measured by an accelerometer of a health monitoring device to adjust an analysis and processing of physiological data measured by other sensors of the health monitoring device is that an impact of motion artifacts on the analysis and processing is reduced.

The disclosure also provides support for a method for determining a state or condition of an organ or organ system of a subject, comprising: using a monitoring device comprising an electrocardiogram (ECG) sensor, an audio sensor, and one or more sensors for measuring a signal that is different from ECG data or audio data to measure said ECG data, said audio data, and said signal from said organ or organ system of said subject, using a trained algorithm to process said ECG data, said audio data, and said signal to determine said state or condition of said organ or organ system of said subject, and providing an output indicative of said state or condition of said organ or organ system of said subject on a computing device. In a first example of the method, the method further comprises: transmitting said ECG data, said audio data, and said signal wirelessly to said computing device. In a second example of the method, optionally including the first example, said monitoring device is a mobile device. In a third example of the method, optionally including one or both of the first and second examples, said computing device is a mobile device. In a fourth example of the method, optionally including one or more or each of the first through third examples, said computing device is part of a cloud system. In a fifth example of the method, optionally including one or more or each of the first through fourth examples, said ECG data, said audio data, and said signal are transmitted in a common packet. In a sixth example of the method, optionally including one or more or each of the first through fifth examples, providing said output indicative of said state or condition of said organ or organ system of said subject comprises a determining a presence or absence of a low ejection fraction of a left ventricle of a heart of said subject. In a seventh example of the method, optionally including one or more or each of the first through sixth examples, said one or more sensors for measuring said signal that is different from said ECG data or said audio data comprises an accelerometer. In an eighth example of the method, optionally including one or more or each of the first through seventh examples, said signal comprises a motion of said monitoring device computed using acceleration from the accelerometer, and wherein using said trained algorithm to process said ECG data, said audio data, and said signal to determine said state or condition of said organ or organ system of said subject comprises: processing said ECG data and said audio data via said trained algorithm responsive to the motion of the monitoring device being less than or equal to a motion threshold, and not processing said ECG data and said audio data via said trained algorithm responsive to the motion of the monitoring device being greater than the motion threshold. In a ninth example of the method, optionally including one or more or each of the first through eighth examples, the method further comprises: transmitting said audio data to a listening device with an audio gain, and reducing the audio gain responsive to the motion of the monitoring device being greater than the motion threshold. In a tenth example of the method, optionally including one or more or each of the first through ninth examples, said signal that is different from said ECG data or said audio data comprises an intrathoracic impedance measurement. In a eleventh example of the method, optionally including one or more or each of the first through tenth examples, said intrathoracic impedance measurement is measured by a same set of electrodes as said ECG data.

The disclosure also provides support for a method for determining a state or condition of a subject, comprising: recording electrocardiogram (ECG) data, audio data, and motion data via sensors of a monitoring device, receiving the ECG data, the audio data, and the motion data from the monitoring device in real-time, processing the received ECG data and the received audio data via an analysis algorithm responsive to the received motion data being less than or equal to a threshold, and not processing the received ECG data and the received audio data via the analysis algorithm responsive to the motion data being greater than the threshold. In a first example of the method, the sensors of the monitoring device include an accelerometer, and the motion data is determined from acceleration measured by the accelerometer. In a second example of the method, optionally including the first example, processing the received ECG data and the received audio data via the analysis algorithm responsive to the received motion data being less than or equal to the threshold comprises: determining an orientation of the monitoring device based on the acceleration measured by the accelerometer, and determining a vector of the received ECG data based on a waveform of the received ECG data and the determined orientation of the monitoring device. In a third example of the method, optionally including one or both of the first and second examples, the method further comprises: transmitting the audio data to a listening device in real-time with a first, higher audio gain responsive to the motion data being less than or equal to the threshold, and transmitting the audio data to the listening device in real-time with a second, lower audio gain responsive to the motion data being greater than the threshold. In a fourth example of the method, optionally including one or more or each of the first through third examples, the analysis algorithm is a cloud-based algorithm trained to determine the state or condition of the subject based on the received ECG data and the received audio data.

The disclosure also provides support for a system for determining a state or condition of a subject, comprising: a communications interface configured to wirelessly communicate with a monitoring device, said monitoring device comprising an electrocardiogram (ECG) sensor, an audio sensor, and at least one other sensor for measuring data from said subject, and a cloud computing network operatively coupled to said communications interface, wherein said cloud computing network is programmed to: receive said data wirelessly from said communications interface in real-time, use a trained algorithm to process said data to determine said state or condition of said subject in real-time, and provide an output indicative of said state or condition of said subject for display on a user interface in real-time. In a first example of the system, said ECG sensor comprises a plurality of electrodes, and wherein said plurality of electrodes are configured to measure both ECG data and intrathoracic impedance data from said subject. In a second example of the system, optionally including the first example, said at least one other sensor comprises an accelerometer, and wherein said cloud computing network is further programmed to: determine which ECG vector is measured by said ECG sensor using knowledge of an orientation of the monitoring device determined from data measured by the accelerometer.

While embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1. A method for determining a state or condition of an organ or organ system of a subject, comprising:
using a monitoring device comprising an electrocardiogram (ECG) sensor, an audio sensor, and one or more sensors for measuring a signal that is different from ECG data or audio data to measure said ECG data, said audio data, and said signal from said organ or organ system of said subject;
using a trained algorithm to process said ECG data, said audio data, and said signal to determine said state or condition of said organ or organ system of said subject; and
providing an output indicative of said state or condition of said organ or organ system of said subject on a computing device.

2. The method of claim 1, further comprising transmitting said ECG data, said audio data, and said signal wirelessly to said computing device.

3. The method of claim 1, wherein said monitoring device is a mobile device.

4. The method of claim 1, wherein said computing device is a mobile device.

5. The method of claim 1, wherein said computing device is part of a cloud system.

6. The method of claim 1, wherein said ECG data, said audio data, and said signal are transmitted in a common packet.

7. The method of claim 1, wherein providing said output indicative of said state or condition of said organ or organ system of said subject comprises a determining a presence or absence of a low ejection fraction of a left ventricle of a heart of said subject.

8. The method of claim 1, wherein said one or more sensors for measuring said signal that is different from said ECG data or said audio data comprises an accelerometer.

9. The method of claim 8, wherein said signal comprises a motion of said monitoring device computed using acceleration from the accelerometer, and wherein using said trained algorithm to process said ECG data, said audio data, and said signal to determine said state or condition of said organ or organ system of said subject comprises:
processing said ECG data and said audio data via said trained algorithm responsive to the motion of the monitoring device being less than or equal to a motion threshold; and
not processing said ECG data and said audio data via said trained algorithm responsive to the motion of the monitoring device being greater than the motion threshold.

10. The method of claim 9, further comprising:
transmitting said audio data to a listening device with an audio gain; and
reducing the audio gain responsive to the motion of the monitoring device being greater than the motion threshold.

11. The method of claim 1, wherein said signal that is different from said ECG data or said audio data comprises an intrathoracic impedance measurement.

12. The method of claim 11, wherein said intrathoracic impedance measurement is measured by a same set of electrodes as said ECG data.

13. A method for determining a state or condition of a subject, comprising:
recording electrocardiogram (ECG) data, audio data, and motion data via sensors of a monitoring device;
receiving the ECG data, the audio data, and the motion data from the monitoring device in real-time;
processing the received ECG data and the received audio data via an analysis algorithm responsive to the received motion data being less than or equal to a threshold; and
not processing the received ECG data and the received audio data via the analysis algorithm responsive to the motion data being greater than the threshold.

14. The method of claim 13, wherein the sensors of the monitoring device include an accelerometer, and the motion data is determined from acceleration measured by the accelerometer.

15. The method of claim 14, wherein processing the received ECG data and the received audio data via the analysis algorithm responsive to the received motion data being less than or equal to the threshold comprises:
determining an orientation of the monitoring device based on the acceleration measured by the accelerometer; and
determining a vector of the received ECG data based on a waveform of the received ECG data and the determined orientation of the monitoring device.

16. The method of claim 13, further comprising:
transmitting the audio data to a listening device in real-time with a first, higher audio gain responsive to the motion data being less than or equal to the threshold; and
transmitting the audio data to the listening device in real-time with a second, lower audio gain responsive to the motion data being greater than the threshold.

17. The method of claim 13, wherein the analysis algorithm is a cloud-based algorithm trained to determine the state or condition of the subject based on the received ECG data and the received audio data.

18. A system for determining a state or condition of a subject, comprising:
a communications interface configured to wirelessly communicate with a monitoring device, said monitoring device comprising an electrocardiogram (ECG) sensor, an audio sensor, and at least one other sensor for measuring data from said subject; and
a cloud computing network operatively coupled to said communications interface, wherein said cloud computing network is programmed to:
receive said data wirelessly from said communications interface in real-time;
use a trained algorithm to process said data to determine said state or condition of said subject in real-time; and
provide an output indicative of said state or condition of said subject for display on a user interface in real-time.

19. The system of claim 18, wherein said ECG sensor comprises a plurality of electrodes, and wherein said plurality of electrodes are configured to measure both ECG data and intrathoracic impedance data from said subject.

20. The system of claim 18, wherein said at least one other sensor comprises an accelerometer, and wherein said cloud computing network is further programmed to:
determine which ECG vector is measured by said ECG sensor using knowledge of an orientation of the monitoring device determined from data measured by the accelerometer.
